(19) United States
(12) Patent Application Publication
(10) Pub. No.: US 2011/0306899 A1
(43) Pub. Date: Dec. 15, 2011

(54) BLOOD DRAWING DEVICE WITH FLASH DETECTION

(75) Inventors: LEROY R. BROWN, Orangevale, CA (US); Lloyd S. Fischel, Haiku, HI (US)

(73) Assignee: ClearView Patient Safety Technologies, LLC, Haiku, HI (US)

(21) Appl. No.: 12/986,864

(22) Filed: Jan. 7, 2011

Related U.S. Application Data
(63) Continuation of application No. 12/822,931, filed on Jun. 24, 2010, now abandoned.
Application No. 12/822,931 is a continuation-in-part of application No. 11/889,536, filed on Aug. 14, 2007.
Application No. 11/889,536 is a continuation-in-part of application No. 10/836,232, filed on May 3, 2004, now Pat. No. 7,396,343.

(51) Int. Cl. A61B 5/153

(52) U.S. Cl. 600/581

(57) ABSTRACT

A device for drawing fluid from a lumen, and particularly blood from a blood vessel, is disclosed. The device may provide indication of the entry of an intravenous cannula into the lumen. The device may include a guide tube for receiving a blood sample container and which may be connected to a needle for drawing blood from a lumen. A rear cannula may extend into the guide tube from the well. A flexible sleeve may be mounted on a barb connected to the guide tube. The flexible sleeve may extend into the guide tube from the well and surround at least a tip portion of the rear cannula. A venting member may be provided between the interior of the sleeve and the ambient. The venting member may permit air to vent from the flexible sleeve without venting blood, so that flash can be detected. In some, but not all, embodiments, one or more passages may be provided in guide tube to facilitate the venting of air from the flexible sleeve.

### CROSS-REFERENCE TO RELATED APPLICATIONS

The present application relates to, is a continuation in part of, and claims the benefit of earlier filed U.S. patent application Ser. No. 11/889,536 which was filed Aug. 14, 2007 and entitled “Blood Drawing Device with Flash Detection,” which relates to, is a continuation in part of, and claims the benefit of earlier filed U.S. patent application Ser. No. 10/836,232 which was filed May 3, 2004 and entitled “Blood Drawing Device with Flash Detection,” and which prior applications are both incorporated herein by reference.

### FIELD OF THE INVENTION

The present invention relates to an apparatus for drawing bodily fluids, and particularly blood, from an animal.

### BACKGROUND OF THE INVENTION

Intravenous blood collection assemblies have long been used to draw bodily fluids, such as blood, from patients. With respect to drawing blood in particular, the vessel or lumen from which the blood is drawn is often rather small and or not visible. If the needle tip is not in communication with the interior of the blood vessel during the procedure, the procedure is likely to be unsuccessful, causing error, undermining the integrity of the specimen, and the patient may be harmed additionally by the penetration of delicate underlying structures. Accordingly, confirmation of accurate placement of the needle tip into a blood vessel is desirable for blood drawing procedures.

Past intravenous blood collection assemblies have included mechanisms for indicating when a needle tip is in communication with the interior of a blood vessel. These needle kits have included a transparent portion in the needle body from which the presence of blood can be observed. The observation of blood in the needle body is known as “flash.” Flash detection has been less than satisfactory for many such collection assemblies. In some instances, the flow of blood into the transparent portion of the needle body is impeded by air backpressure in the needle, and thus flash confirmation is not visible or delayed. This delay can impede the determination of the precise moment at which the needle tip enters the blood vessel, which may cause the healthcare worker inserting the needle to miss or perforate the vessel and penetrate into delicate surrounding structures. In other instances, while flash occurs, the visual indication of flash is not easily detected because the amount of flash is small or obscured due to the positioning of the collection assembly. Accordingly, there is a need for a blood-drawing device that provides flash relatively rapidly and to an extent that a user may readily detect it.

There are also many needles that are currently used that do not provide for flash detection. It would be advantageous to be able to retrofit such needles so that they may be used to draw blood and provide detection of flash.

### SUMMARY OF THE INVENTION

Responsive to the foregoing challenges, Applicant has developed a guide tube assembly for connection to a device for drawing fluid from a lumen, comprising: a guide tube (116) having an open end (118) and a well (117); a flexible sleeve (150, 151) having an interior space in fluid communication with the well (117) and extending into the guide tube (116); one or more passages (332, 334, 336, 338) extending through the guide tube (116) which are in fluid communication with the flexible sleeve (150) interior space; and a venting member (160) connected to the guide tube (116) or incorporated into the flexible sleeve (150, 151), said venting member in fluid communication with the one or more passages (332, 334, 336, 338) and disposed between the flexible sleeve (150, 151) interior space and an ambient.

It is to be understood that both the foregoing general description and the following detailed description are exemplary and explanatory only, and are not restrictive of the invention as claimed.

### BRIEF DESCRIPTION OF THE DRAWINGS

In order to assist the understanding of this invention, reference will now be made to the appended drawings, in which like reference characters refer to like elements.

FIG. 1 is a side view in cross-section of a blood drawing needle and guide tube having a venting member in accordance with a first embodiment of the present invention prior to connection with a blood drawing needle.

FIG. 2 is a side view in cross-section of the guide tube shown in FIG. 1 after connection to the blood drawing needle.

FIG. 3 is a side view in cross-section of a guide tube having a venting member in accordance with an second embodiment of the present invention prior to connection with a blood drawing needle.

FIG. 4 is an end view of the raised dimple surface of a well bottom wall provided in the guide tube in accordance with an alternative embodiment of the invention shown in FIGS. 1-3.

FIG. 5 is an end view of the raised radial line surface of a well bottom wall provided in the guide tube in accordance with an alternative embodiment of the invention shown in FIGS. 1-3.

FIG. 6 is a side view in cross-section of a blood drawing device with a guide tube having a venting member located in a guide tube opening in accordance with a third embodiment of the present invention.

FIG. 7 is a side view in cross-section of a blood drawing device with a guide tube having a venting member located in a guide tube opening with well bottom extensions surrounding the venting member in accordance with a fourth embodiment of the present invention.

FIG. 8 is a side view in cross-section of a blood drawing device with a guide tube having a venting member and viewing window located in a guide tube side wall in accordance with a fifth embodiment of the present invention.

FIG. 9 is a side view in cross-section of a blood drawing device with a guide tube having a venting member and viewing window located in a guide tube side wall in accordance with a sixth embodiment of the present invention.

FIG. 10 is a side view in cross-section of a blood drawing device with a guide tube having a venting member located in a guide tube opening in accordance with a seventh embodiment of the present invention.

FIG. 11 is a side view in cross-section of a blood drawing device with a guide tube having a venting member located in a guide tube opening and a viewing window located in a guide tube side wall in accordance with an eighth embodiment of the present invention.

FIG. 12 is a side view in cross-section of a blood drawing device with a guide tube having a venting member located in a guide tube opening surrounded by well bottom extensions and having a viewing window located in a guide tube side wall in accordance with a ninth embodiment of the present invention.

FIG. 13 is a side view in cross-section of a blood drawing device with a guide tube having a venting member located in a guide tube opening with well bottom extensions surrounding the venting member in accordance with a tenth embodiment of the present invention.

FIG. 14 is a side view in cross-section of a blood drawing device with a guide tube having a sealing membrane in a guide tube well and a venting member and viewing window located in a guide tube side wall in accordance with an eleventh embodiment of the present invention.

FIG. 15 is a side view in cross-section of a blood drawing device with a guide tube having a venting member and viewing window located in a guide tube side wall in accordance with a twelfth embodiment of the present invention.

FIG. 16 is a side view in cross-section of a blood drawing device with a guide tube having a venting member located in a guide tube well in accordance with a thirteenth embodiment of the present invention.

FIG. 17 is a side view in cross-section of a blood drawing device with a guide tube having a venting member located in a sleeve tubulation and a viewing window located in a guide tube side wall in accordance with a fourteenth embodiment of the present invention.

FIG. 18 is a side view in cross-section of a blood drawing device with a guide tube having an air-permeable sleeve and a viewing window located in a guide tube side wall in accordance with a fifteenth embodiment of the present invention.

FIG. 19 is a side view in cross-section of a blood drawing device with a guide tube having a sleeve with a venting portion and a viewing window located in a guide tube side wall in accordance with a sixteenth embodiment of the present invention.

FIG. 20 is a side view in cross-section of a blood drawing device with a guide tube having a venting member located in a sleeve tubulation and a viewing window located in a guide tube side wall in accordance with a seventeenth embodiment of the present invention.

FIG. 21 is a side view in cross-section of a blood drawing device with a guide tube having an air-permeable sleeve and a viewing window located in a guide tube side wall in accordance with an eighteenth embodiment of the present invention.

FIG. 22 is a side view in cross-section of a blood drawing device with a guide tube having a sleeve with a venting portion and a viewing window located in a guide tube side wall in accordance with a ninteenth embodiment of the present invention.

FIG. 23 is a side view in partial cross-section of a first Luer-adapter type butterfly needle attachment which may be used to connect with the guide tubes shown in FIGS. 1-22.

FIG. 24 is a side view in cross-section of a second butterfly needle attachment that may be used to directly connect to the guide tubes shown in FIGS. 1-22.

### DETAILED DESCRIPTION OF EMBODIMENTS OF THE INVENTION

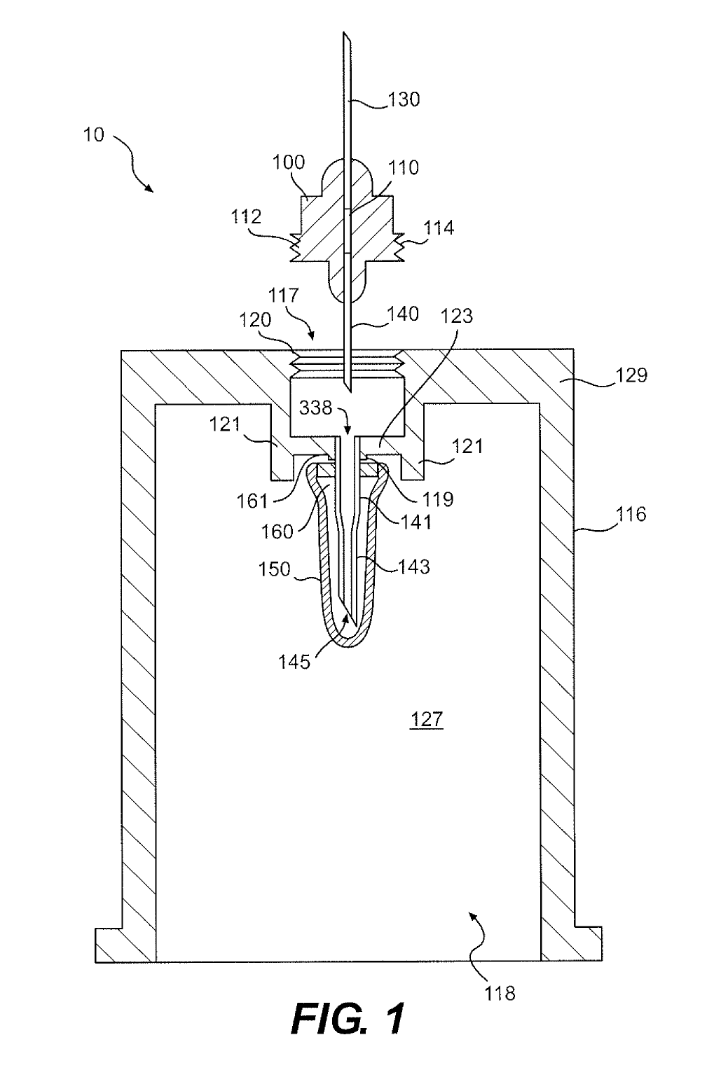

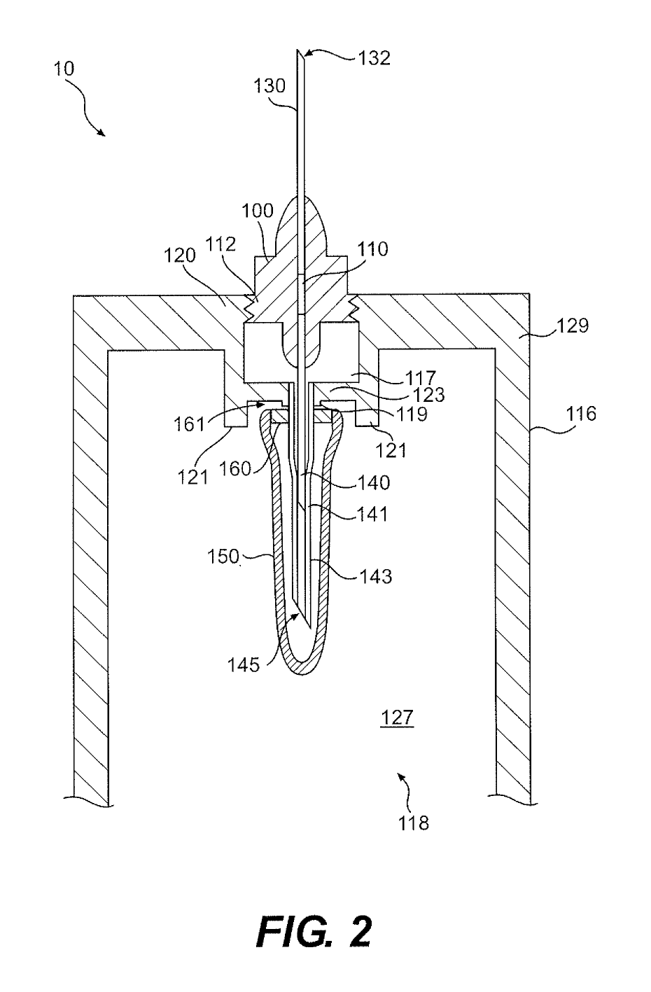

Reference will now be made in detail to a first embodiment of the present invention, an example of which is illustrated in the accompanying drawings. With reference to FIGS. 1 and 2, a guide tube 116 is shown. The guide tube 116 is provided for connection to a central body 100 which may include one or more constituent elements, such as a threaded connector 112 with interlocking threads 114 or other connection means, for connecting the central body to the guide tube. The threaded connector 112 may be integrally formed with, or connected to the central body using adhesive, male-female interfaces, threaded interfaces, or any other connection means. It is appreciated that the guide tube 116 may be connected to the central body 100 by any connection means, including without limitation the threaded interfaces shown, male-female interfaces, pressure-fit connectors, slot and key connectors, snap-on connectors, and the like. The type of connection between the guide tube 116 and the central body 100 is not intended to be limiting to the scope or use of any of the present embodiments of the invention.

A fluid passage 110 within the central body 100 may communicate with, and in the embodiment shown, be connected to the front cannula 130 and the first rear cannula 140, respectively, using adhesive, threaded interfaces, pressure fit, or other connection means. Alternatively, the central body 100 may be integrally formed with the front and/or first rear cannulae 130 and 140. It is further appreciated the front and first rear cannulae 130 and 140 may be formed of a continuous material and eliminate the need to fluid passage 110. It is also appreciated that the front and/or rear cannulae may be transparent or translucent, in whole or part, to provide flash detection in alternative embodiments of the present invention. If included, the fluid passage 110 may be defined by the opening within the central body between the front and rear cannulae when the cannulae are directly connected to the central body. The fluid passage 110 may be adapted to receive a sufficient amount of fluid to allow observation of the fluid (i.e., “flash”) from outside the blood-drawing device 10. At the same time, the fluid passage 110 may have a sufficiently small volume so as to rapidly fill with fluid during the use of the blood-drawing device.

Preferably, the central body 100 may be constructed of plastic material suitable for medical use. Further, in any embodiment of the present invention, all, or portions, of the central body 100 may be transparent, translucent, connected to transparent or translucent I.V. tubing, or otherwise adapted to permit detection of fluids passing through the central body and/or I.V. tubing from a vantage point outside of the blood-drawing device 10. For example, with reference to FIG. 1, the central body 100 may include a transparent wall that is adapted to permit the observation of “flash” when it occurs. In an alternate embodiment of the present invention, the side wall of the central body 100 also may be adapted to magnify or otherwise enhance the detection of fluid passing through the central body, although it is appreciated that a magnifying or enhancement feature is not necessarily required.

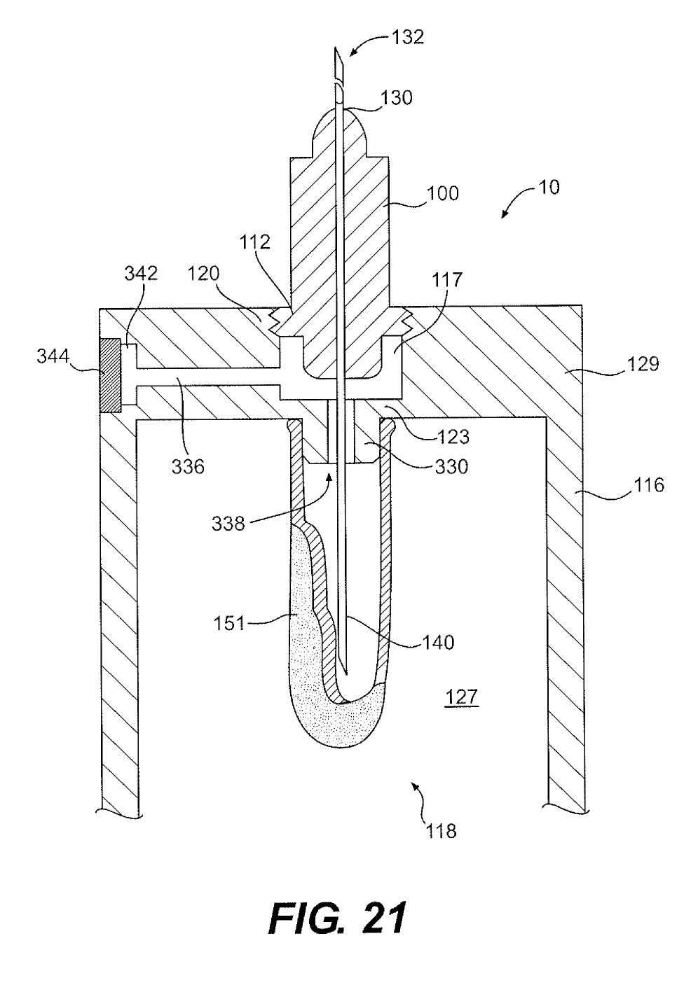

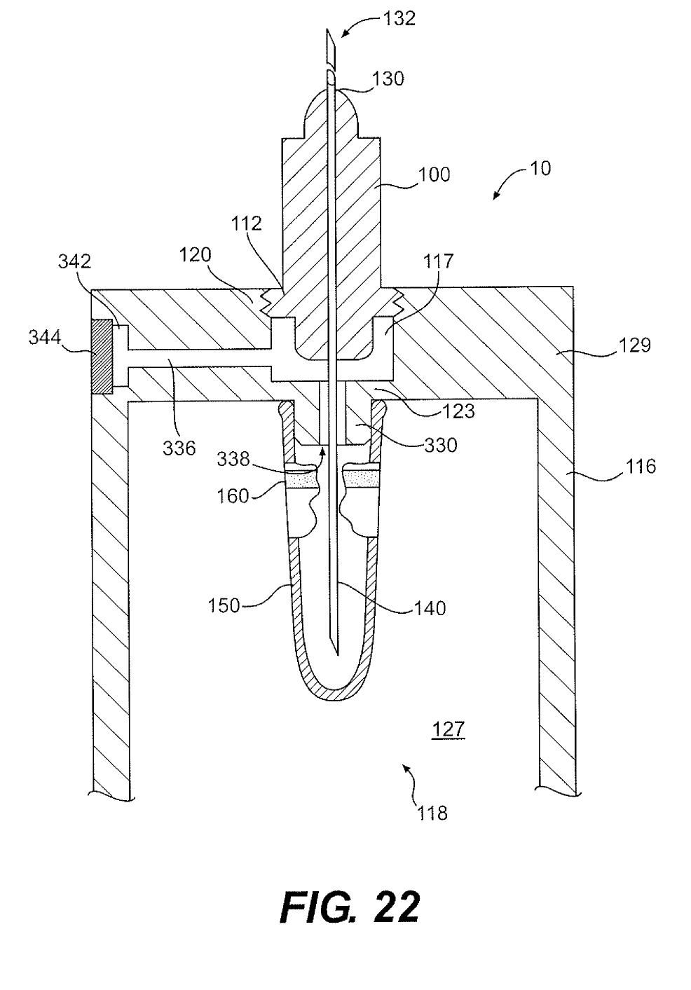

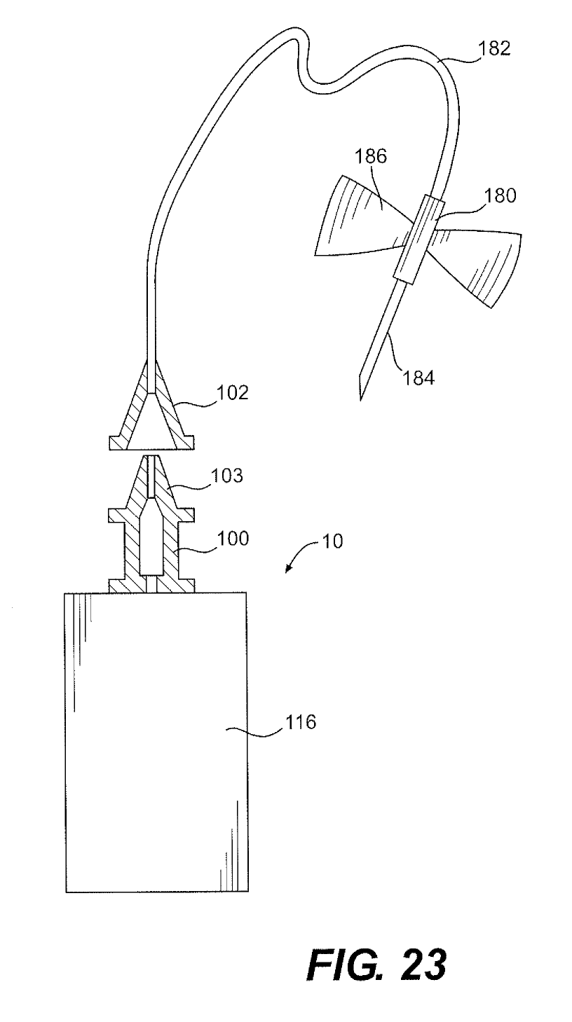

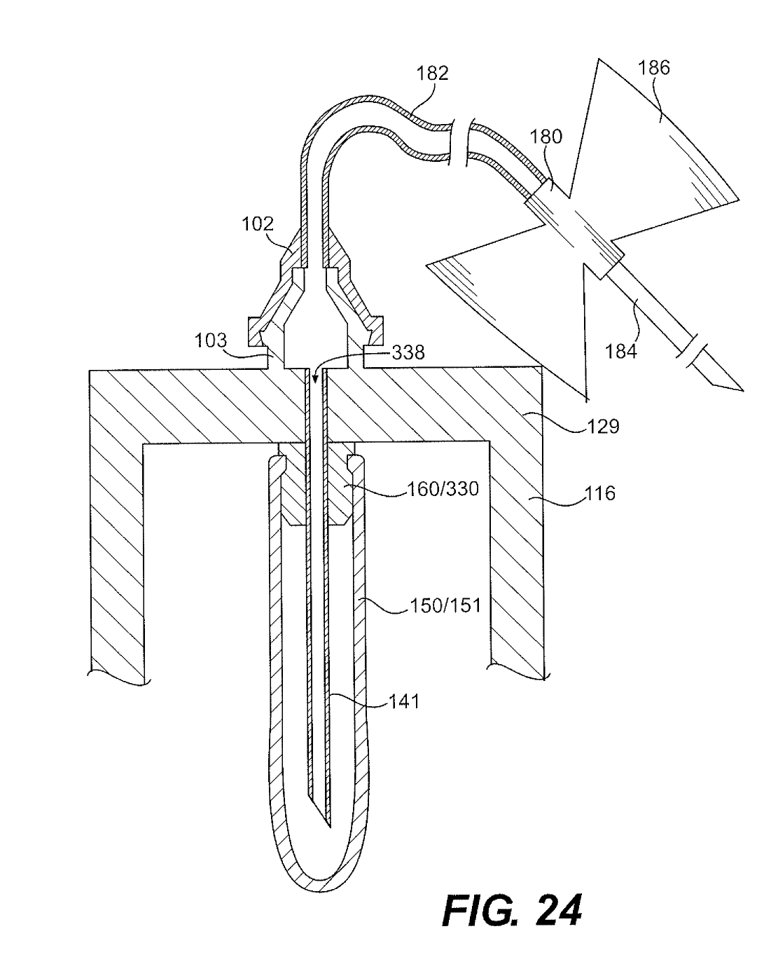

It is further appreciated that the front cannula 130 may be replaced with or connected to a flexible butterfly connection tube 182 that is part of a butterfly needle 180 as shown in FIG. 23. When connected to a flexible butterfly connection tube 182, the front cannula 130 extending from the central body 100 may be eliminated and replaced with a Luer-adapter including a female connector 102 and male connector 103 which is connected via the butterfly connection tube 182 to a butterfly cannula 184 which substitutes for the front cannula 130. The flexible butterfly connection tube 182 may be translucent or transparent when combined with the embodiments shown in FIGS. 1-22 so that the “flash” of blood that enters the device 10 may be visible first in the butterfly connection tube. In a further alternative, shown in FIG. 24 and applicable to the embodiments shown in FIGS. 1-22, the female connector 102 of the Luer-adapter may connect directly to a specially formed mating male connector 103 integrally formed with the guide tube 116. It is appreciated that the placement of the male and female connectors could be reversed or replaced with other connector types in alternative embodiments of the invention.

With renewed reference to FIGS. 1-2, the front cannula 130 may extend from the front end of the central body 100 and terminate at a tapered or pointed end 132 which is adapted to be inserted into a lumen. The first rear cannula 140 may extend from the rear of the central body 100 and terminate at a tapered or pointed end.

A generally cylindrical guide tube 116 is adapted to be connected to the central body 100. Preferably, the guide tube 116 may be made in whole or part of transparent or translucent material that permits viewing of the interior space of the guide tube. The guide tube 116 may include a substantially closed end 129, an open end 118 at one end communicating with an interior opening 127, and a well 117 at the opposite end defined in part by a well bottom wall 123. The open end 118 may be adapted to receive a blood or fluid sample container. The upper edge of the well 117 may include threads or other connection means 120 adapted to mate with the threads 114 of the threaded connector 112 provided on the central body 100.

The well bottom wall 123 may include a centrally located opening through which a second rear cannula 141 extends towards the guide tube open end 118. The second rear cannula 141 may be sufficiently fastened to the well bottom wall 123 by any means such that the application of pressure from a fluid sample container pressed against the second rear cannula will not dislodge the second rear cannula. The guide tube 116 may extend coaxially with the second rear cannula 141 sufficiently beyond the tapered end 145 to provide some degree of protection against inadvertent “needle sticks” by a user of the blood-drawing device 10 as well as to guide the reception of a fluid sample container.

The bottom surface of the well bottom wall 123 located within the interior of the guide tube 116 may include a shoulder 119 against which a venting member 160 may be disposed. The shoulder 119 may create an air space 161 between the bottom surface of the well 117 and the venting member 160. In one alternative embodiment of the invention, shown in FIG. 4, the shoulder 119 may be replaced by or incorporate a surface having raised dimples 217, or in another alternative embodiment, as shown in FIG. 5, the shoulder may be replaced by or incorporate a surface having raised radial lines 219, or some other surface which both supports the venting member 160 and provides for air flow between the venting member and the interior of the guide tube 116. In still other alternative embodiments of the present invention, an air-permeable spacer may be provided between the venting member 160 and the bottom surface of the well bottom wall 123 to provide the required air flow in place of any raised features on the bottom of the well bottom wall.

The second rear cannula 141 may include a stepped diameter 143 or other narrowing feature that results in the second rear cannula having a narrower diameter at the pointed end 145 compared with the end extending from the well 117. The end of the second rear cannula 141 nearest to the well 117 may have a diameter sufficient to receive the first rear cannula 140 within it. In alternative embodiments of the invention, the second rear cannula 141 may have a uniform diameter over its entire length.

One or more well bottom extensions 121 may also extend from the well bottom wall 123 into the guide tube opening 118 adjacent to the venting member 160. The well bottom extensions 121 may surround the venting member 160 in whole or in part and may be formed so as to leave an air space for air to flow from the interior of the sleeve 150 through the venting member 160 to the ambient in the guide tube interior opening 127. The well bottom extensions 121 may be semi-circular, or prong shaped, and surround the venting member 160. Further, the well bottom extensions 121 may extend into the guide tube 116 a distance sufficient to protect the venting member 160 from being impacted against, damaged or dislodged by, the stopper of a container inserted into the guide tube. The well bottom extensions 121 may be formed of plastic material which is integral with the rest of the guide tube 116 and well bottom wall 123. The well bottom extensions 121 may also contact the venting member 160 and/or flexible sleeve 150 so as to assist in retaining the sleeve and venting member in the guide tube 116.

The venting member 160 may be constructed out of any material which is capable of substantially preventing blood from passing through it while permitting air to vent through it. Preferably, for purposes of ease of manufacturing, the venting member 160 may be formed as a stamped disk with a central opening from a sheet of venting member material for the embodiment shown in FIGS. 1-2.

The venting member 160 may be constructed of any of a number of materials that provide the desired level of porosity, which may include, but are not limited to sintered, layered, rolled, foamed, perforated, or impregnated, hydrophyllic/hydrophobic compositions, porous polyethylene, porous polypropylene, porous polyfluorocarbon, absorbent paper, materials impregnated with dilute Russell Viper venom molded fiber, fiberglass, felt, granular starch, cellulose, polyacrylamide gel, hydrogel, a molded admixture of porous hydrophobic/hydrophyllic granules and sufficiently low density silicone, molded open cell polyurethane, and like polymeric materials. Examples of materials that may be used to construct the venting (i.e., porous) member 160 are discussed in U.S. Pat. No. 4,207,870 to Eldridge, and U.S. Pat. No. 4,340,068 to Kaufman, each of which are hereby incorporated by reference.

The venting member 160 (i.e., a means for venting air) may be inserted over the second rear cannula 141 and pressed against or near to the rear portion of the well bottom wall 123. The venting member 160 may form a seal against the second rear cannula 141 that is sufficient to prevent blood from escaping past the venting member. In the embodiments of the present invention, the venting member 160 may be gas, and particularly air, permeable, but at least partially, and preferably wholly, impermeable to a liquid, such as blood. Preferably, the venting member 160 may be substantially porous for gas constituents less than about 5 microns in size, and substantially non-porous for liquid constituents about 5 microns or greater in size, however, it is appreciated that these approximate sizes should not be limiting for the invention.

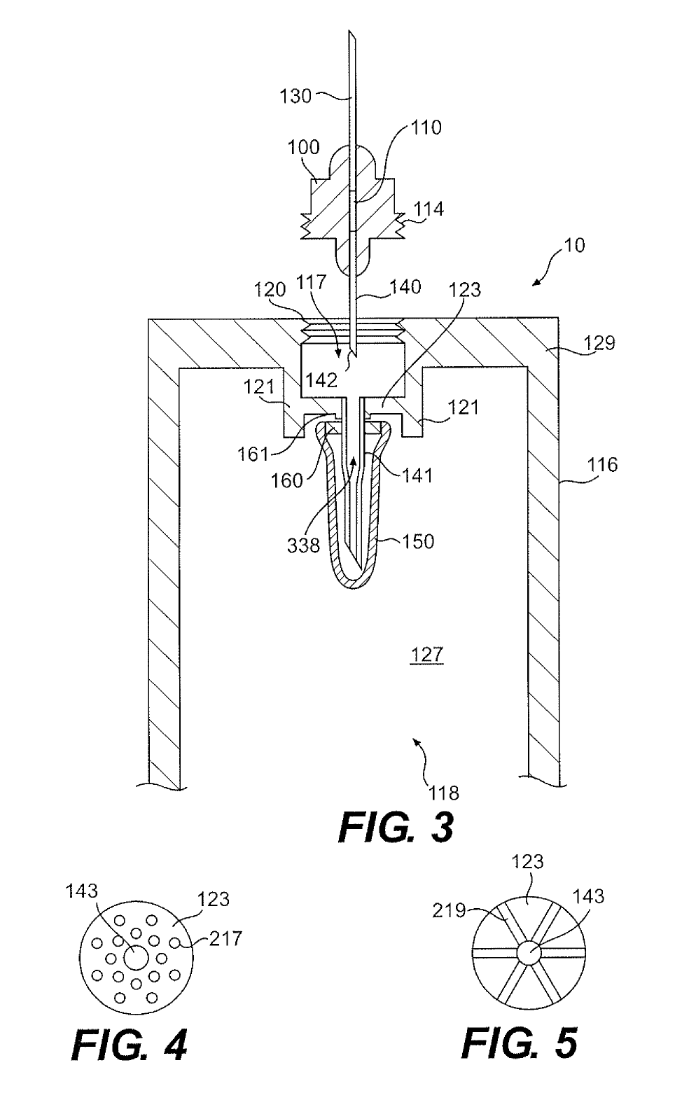

For each of the embodiments discussed in connection with FIGS. 3-24, the venting member 160 may be constructed in accordance with the description of the venting members described in connection with the embodiment of the invention described in connection with FIGS. 1-2.

A flexible sleeve 150 may be disposed over and around the second rear cannula 141. The sleeve 150 may isolate the second rear cannula 141 from the ambient, wherein the ambient includes any space outside of the sleeve 150, irrespective of whether or not the space is contained within the guide tube 116 or any other structure. The flexible sleeve 150 may be stretched over all or part of the side wall of the venting member 160, or in alternate embodiments, otherwise contact the venting member 160. The flexible sleeve 150 may be made of a shape memory material, such as elastic rubber or elastomeric silicone or latex, or the like, which will return to the shape shown in FIG. 1 as long as no other structure obstructs it. Examples of materials that may be used to construct the flexible sleeve 150 are discussed in U.S. Pat. No. 3,877,465 to Miyake, U.S. Pat. No. 5,086,780 to Schmitt, U.S. Pat. No. 6,110,160 to Farber, U.S. Pat. No. 6,533,760 to Leong, U.S. Patent Pub. No. US 2002/0004647 A1 to Leong, and U.S. Patent Pub. No. US 2003/0078544 A1 to Chen, each of which is hereby incorporated by reference. It is appreciated that any suitable material may be used for the flexible sleeve without departing from the intended scope of the present invention.

The flexible sleeve 150, also known as a multiple sample sleeve, may preferably be transparent or translucent such that the presence of blood within the flexible sleeve may be visually detected. When the flexible sleeve 150 is transparent or translucent in any of the embodiments shown in FIGS. 1-24, the guide tube 116 may also be transparent or translucent so that the “flash” of blood may be visible through both the guide tube and the flexible sleeve to the person drawing blood. The length of the flexible sleeve 150 may be sufficient to accommodate the second rear cannula 141 but not so long as to present serious risk of the second rear cannula piercing the side wall of the flexible sleeve when a fluid sample container is pushed into the guide tube 116.

Prior to using the device 10 to draw blood, the central body 100 may be connected to the guide tube 116 at the threaded end 112 as shown in FIG. 2, in which like reference characters refer to like elements. When so connected, the combination of the guide tube 116 with a venting member 160 and the flexible sleeve 150 may be used to effectively “retrofit” a non-venting blood drawing device to become a venting blood drawing device. It is appreciated that each of the embodiments of the present invention shown in FIGS. 1-24 may be used to retrofit existing non-venting blood drawing devices to become devices which vent air without substantially venting blood. The well 117 may be designed to provide sufficient space to accommodate the rear portion of the central body 100 of a conventional or known blood drawing device within it to provide retro-fitting.

The function of the first embodiment of the blood-drawing device 10 will now be described with reference to FIGS. 1-2. With reference to FIGS. 1-2, the tapered end 132 of the front cannula 130 (or some extension thereof) may be inserted into a fluid containing body lumen prior to the insertion of a fluid sample container into the guide tube 116. In a preferred embodiment of the present invention, the front cannula 130 is inserted into a lumen containing a visually detectable fluid, such as blood. At the time that the front cannula 130 is inserted into the body lumen, it is assumed that the internal passages within the blood-drawing device (i.e., the passage through the front cannula 130, the fluid passage 110, the passage through the rear cannula 140, and the space inside the flexible sleeve 150) may be filled with atmospheric air or some other gas. When the front cannula 130 establishes communication with the fluid in the body lumen, fluid pressure in the lumen may force the fluid through the front cannula 130 towards the fluid passage 110.

The flow of fluid through the front cannula may begin to compress the air in the fluid passage 110, the rear cannula 140, the second rear cannula 141, and the space between the rear cannulae and the flexible sleeve 150, driving the air towards the venting member 160. Air within the blood drawing device 10 that would otherwise prevent or slow the flow of blood into it, may vent through the flexible sleeve 150 and the venting member 160 to the ambient within the guide tube 116. The air may vent from the venting member 160 through the air space 161 that may be created by the shoulder 119 or the dimples 217 (FIG. 4) or radial lines 219 (FIG. 5). As a result, there may be insufficient air pressure within the fluid passage 110 to resist the flow of the fluid, where it may be detected or observed as “flash” by a user.

Blood that flows into the blood drawing device may be viewed either through the central body 100, the butterfly connection tube 182 (in embodiments that use a butterfly needle) or through a transparent or translucent flexible sleeve 150 and/or a transparent or translucent guide tube 116. The visual detection of blood indicates positive vein entry, known as flash. It is appreciated that “flash” may be detected at any point along the device that includes a transparent or translucent member, which may include, but not be limited to, a transparent or translucent cannula, central body, I.V. tubing, flexible sleeve, or other constituent member.

Once flash is detected, a fluid sample container may be pressed into the open end 118 of the guide tube 116, the second rear cannula 141 may pierce the flexible sleeve 150, and the flexible sleeve is pushed back towards the base of the second rear cannula. As shown in FIG. 2, the first rear cannula 140 may slide into the second rear cannula 141 and the second rear cannula may extend into the guide tube 116 a distance which is sufficient to pierce the stopper provided on the top of a fluid sample container. The one or more well bottom extensions 121 may prevent the fluid sample container from crushing, dislodging or otherwise damaging the venting member 160. A sample of blood may then be received in the fluid sample container.

An alternative embodiment of the present invention is shown in FIG. 3, in which like reference characters refer to like elements in the other drawing figures. The embodiment shown in FIG. 3 differs from that shown in FIGS. 1-2 in that the second rear cannula 141 is truncated. In this embodiment the first rear cannula 140 is of sufficient length to pierce a fluid sample container. It is also appreciated that the second rear cannula 141 could be replaced with an integral portion of the well bottom wall 123 shaped to receive the venting member 160. In all other respects the embodiment shown in FIG. 3 operates like the embodiment shown in FIGS. 1-2.

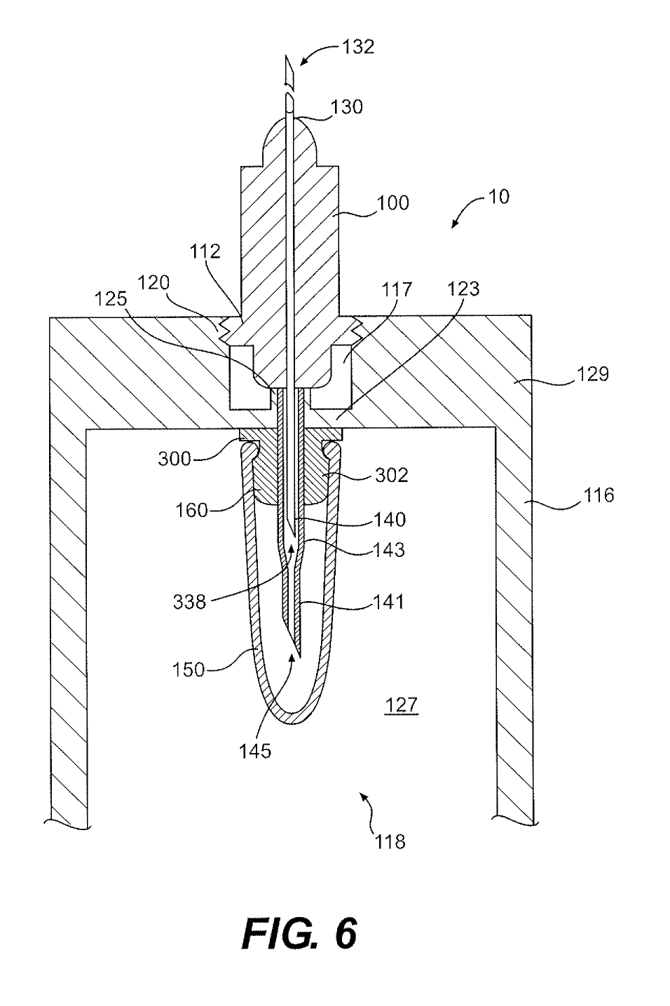

With reference to FIG. 6, another embodiment of the present invention is shown in which all reference characters refer to like elements shown in the previous drawing figures. The well bottom wall upper surface 125 may prevent blood from flowing into the well 117 when the central body 100 is seated against the well bottom wall 123.

With continued reference to FIG. 6, the second rear cannula 141 may have a venting member 160 disposed about it at a base portion against the inner end wall of the guide tube 116. The venting member 160 may have a base 300 and a barb portion 302. The base 300 may be adhered to the end wall of the guide tube 116, pressure fit onto the second cannula 141, or otherwise fixed into position within the guide tube 116 without being directly connected to the central body 100 or any element of the central body, such as the first rear cannula 140. The venting member 160 base 300 may be defined in part by an annular recess which separates the base from the barb portion 302 that is adapted to have the open end of a flexible sleeve 150 mounted thereon. Alternatively, the venting member 160 need not have any annular recess so long as the flexible sleeve 150 has a base portion 300 which provides an air path for air to flow from inside the flexible sleeve 150 to the interior of the guide tube 116. The air path may be provided in whole or in part by well bottom wall 123 features such as dimples 217 (FIG. 4), radial lines 219 (FIG. 5) or the like. The embodiment shown in FIG. 6, as well as the embodiments of the invention shown in FIGS. 1-5 and 7-24, may be used to retrofit an existing and non-air venting needle central body 100 into an air venting blood drawing device after being connected to the guide tube 116.

With continued reference to FIG. 6, when the front cannula 130 (or butterfly cannula 184 with respect to FIGS. 23-24) is introduced to a lumen, such as a vein, to draw fluid, such as blood, the blood may readily flow through the front cannula, the first rear cannula 140, the second rear cannula 141 and into the interior of the flexible sleeve 150. Air within the blood drawing device 10 that would otherwise prevent or slow the flow of blood into it, may vent through the flexible sleeve 150 and the venting member 160 through the venting member base 300 to the ambient within the guide tube 116. The venting member base 300 may provide a surface for air to escape from the venting member 160. Blood that flows into the blood drawing device may be viewed either through the central body 100, the butterfly connection tube 182 (in embodiments that use a butterfly needle) or through a transparent or translucent flexible sleeve 150 and transparent or translucent guide tube 116. The visual detection of blood indicates positive vein entry, known as flash. Once flash is detected, a fluid sample container may be pressed into the open end 118 of the guide tube 116, the second rear cannula 141 may pierce the flexible sleeve 150 and the flexible sleeve is pushed back towards the base of the second rear cannula. The shape of the venting member 160 with a wide base 300 and barb portion 302 may be such as to withstand the force of a fluid sample container being inserted into the guide tube and against the venting member without the venting member being crushed or otherwise damaged. This is necessary so that multiple sample containers can be filled with blood without the risk of leakage past a damaged venting member.

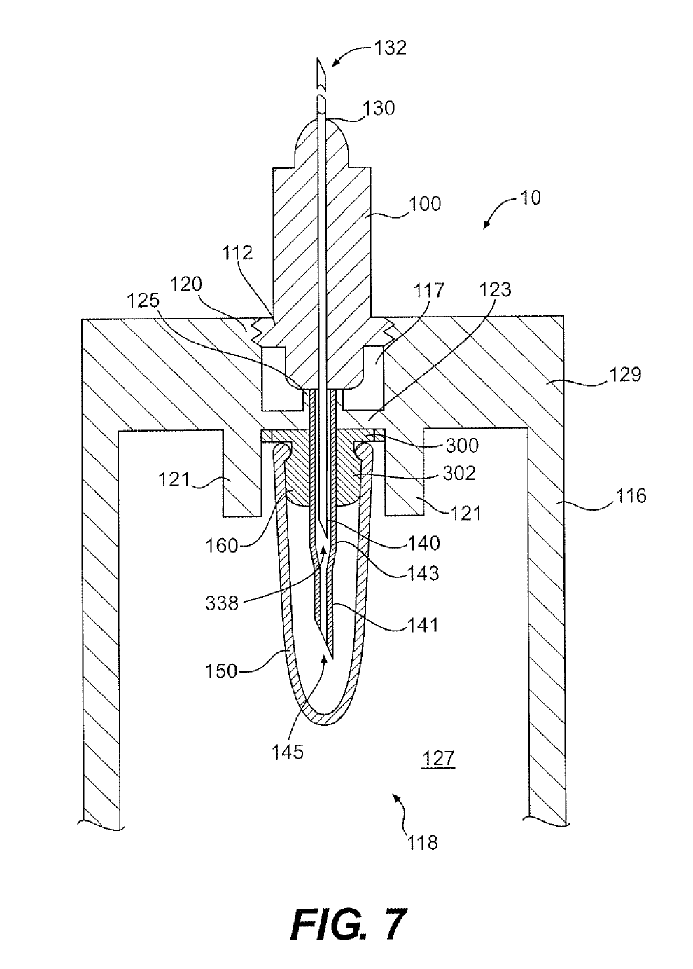

With reference to FIG. 7, another embodiment of the present invention is shown in which all reference characters refer to like elements shown in the previous drawing figures. The embodiment shown in FIG. 7 is identical in all respects to that shown in FIG. 6 with the following exception. With respect to FIG. 7, one or more well bottom extensions 121 may extend from the well bottom wall 123. The well bottom extensions 121 may surround the venting member 160 in whole or in part and may be formed so as to leave an air space for air to flow from the interior of the sleeve 150 through the venting member 160 to the ambient in the guide tube opening 118. The well bottom extensions 121 may be semi-circular or prong shaped and surround the venting member 160. Further, the well bottom extensions 121 may extend into the guide tube 116 a distance sufficient to protect the venting member 160 from being impacted against, damaged or dislodged by, the stopper of a container inserted into the guide tube. The well bottom extensions 121 may be formed of plastic material which is integral with the rest of the guide tube 116 and well bottom wall 123. The well bottom extensions 121 may also be used to secure the venting member 160 and more particularly, the base portion 300 of the venting member so that the venting member is retained in place without the need to use an adhesive or other connection means to the guide tube 116.

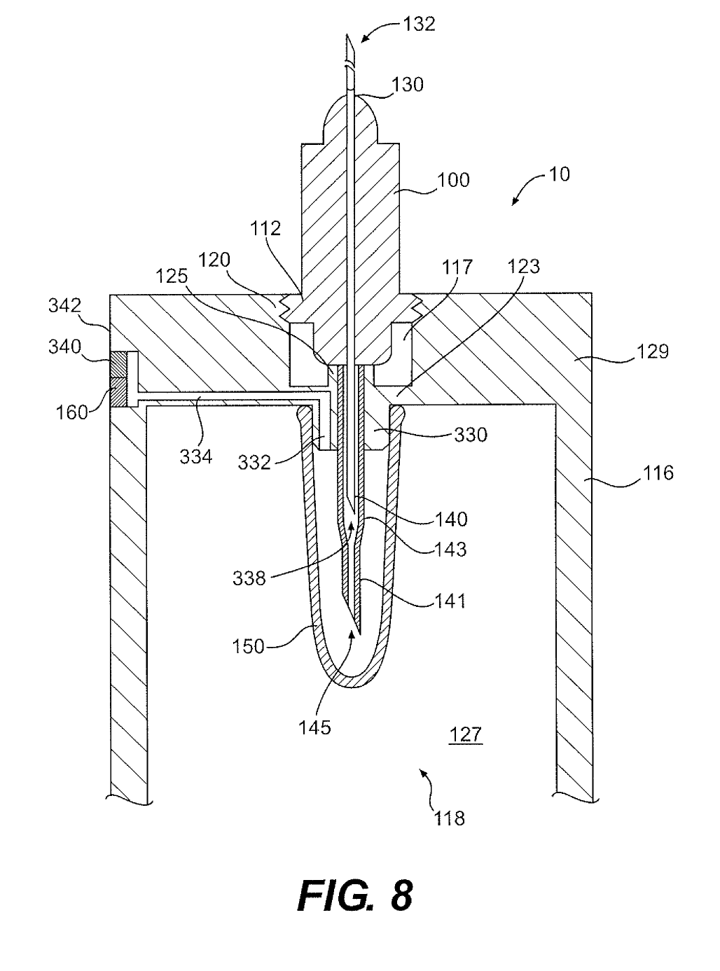

With reference to FIG. 8, another embodiment of the present invention is shown in which all reference characters refer to like elements shown in the previous drawing figures. As noted above with respect to FIGS. 6-7, the well bottom wall upper surface 125 may prevent blood from flowing into the well 117 when the central body 100 is seated against the well bottom wall 123.

With continued reference to FIG. 8, the flexible sleeve 150 may be mounted or fitted onto a sleeve connector 330 which is integrally formed with the guide tube 116 within the guide tube opening 118. A sleeve connector passage 332 may extend through the sleeve connector 330 and connect with a lateral passage 334 that extends from the sleeve connector passage 332 to a guide tube aperture 342 located along the guide tube outer wall. The guide tube aperture 342 may have a venting member 160 disposed in it, and an optional viewing window 340 disposed in it. The viewing window 340 may be transparent or translucent and may permit blood to be viewed by a user through the viewing window so as to detect flash. The viewing window 340 may be disposed next to, circumferentially about, or within the venting member 160 without departing from the intended scope of the invention. In fact, the viewing window 340 and the venting member 160 may be disposed in separate apertures in alternative embodiments. The viewing window 340 also may be unnecessary if flash is detected elsewhere, such as through a translucent or transparent butterfly connection tube, central body, flexible sleeve and/or guide tube.

The blood drawing device shown in FIG. 8 may be used as follows. The central body 100 may be connected to the guide tube 116 such that the rear portion of the central body seals against the well bottom wall upper surface 125. Such sealing is not required in this or any other embodiments discussed, but is preferred to limit the amount of blood that may flow into the well space 117 if the central body 100 is disconnected from the guide tube 116.

Once the central body 100 is connected to the guide tube 116, the front cannula 130 or butterfly cannula 184 may be introduced into a lumen, such as a vein, to draw fluid, such as blood. The blood may readily flow through the front cannula 130, the first rear cannula 140, the second rear cannula 141 and into the interior of the flexible sleeve 150. Air within the blood drawing device 10 that would otherwise prevent or slow the flow of blood into it, may vent through the flexible sleeve 150, the sleeve connector passage 332, the lateral passage 334, and the venting member 160 to the ambient outside the guide tube 116. Blood that flows into the blood drawing device may be viewed either through the central body 100, the butterfly connection tube 182 (in embodiments that use a butterfly needle), through a transparent or translucent flexible sleeve 150 and transparent or translucent guide tube 116, or through the optional viewing window 340. The visual detection of blood indicates positive vein entry, known as flash. Once flash is detected, a fluid sample container may be pressed into the open end 118 of the guide tube 116, the second rear cannula 141 may pierce the flexible sleeve 150 and the flexible sleeve is pushed back towards the base of the second rear cannula.

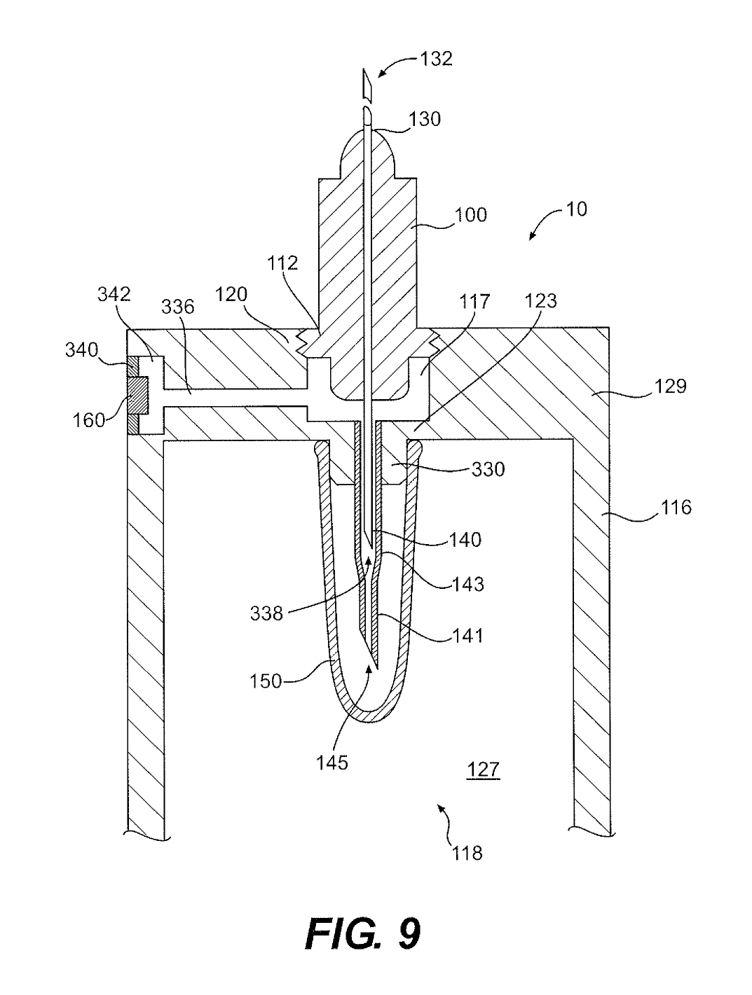

With reference to FIG. 9, another embodiment of the present invention is shown in which all reference characters refer to like elements shown in the previous drawing figures. The embodiment shown in FIG. 9 is identical in all respects to that shown in FIG. 8 with the following exception. With respect to FIG. 9, a well passage 336 extends from the well 117 to the aperture 342. Air may vent from the rear cannula 140 up through the second rear cannula 141 and into the well 117. The air may then flow through the well passage 336 and the venting member 160. Blood may fill the well passage 336 and the aperture 340. Blood may be viewed either through the central body 100, the butterfly connection tube 182 (in embodiments that use a butterfly needle), through a transparent or translucent flexible sleeve 150 and transparent or translucent guide tube 116, or through the optional viewing window 340.

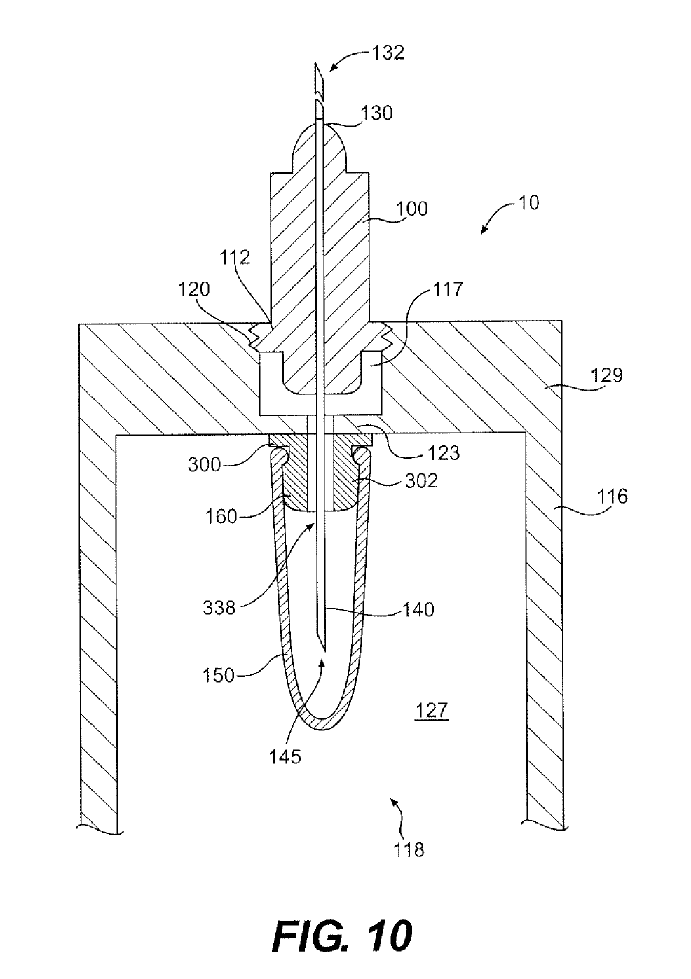

With reference to FIG. 10, another embodiment of the present invention is shown in which all reference characters refer to like elements shown in the previous drawing figures. A venting member 160 may be disposed against the well bottom wall 123 of the guide tube 116. The venting member 160 base portion 300 may be adhered to the well bottom wall of the guide tube 116, pressure fit onto the cannula 140, or otherwise fixed into position. A central well opening 338 may extend through the venting member 160 and the well bottom wall 123 to permit air and blood to flow into the well 117. Alternatively, the central well opening 338 may be reduced in diameter so that the cannula 140 seals against the well bottom wall 123 and/or the venting member 160. The venting member 160 may have a base 300 separated from a barb portion 302 by an annular recess. The barb portion 302 may be adapted to have an open end of a flexible sleeve 150 mounted thereon. Alternatively, the venting member 160 need not have any annular recess so long as the flexible sleeve 150 has a base portion 300 which provides an air path for air to flow from inside the flexible sleeve 150 to the interior of the guide tube 116. The air path may be provided in whole or in part by well bottom wall 123 features such as dimples 217 (FIG. 4), radial lines 219 (FIG. 5) or the like.

With continued reference to FIG. 10, when the front cannula 130 or butterfly cannula 184 is introduced to a lumen, such as a vein, to draw fluid, such as blood, the blood may readily flow through the front cannula, the first rear cannula 140 and into the interior of the flexible sleeve 150. Air within the blood drawing device 10 that would otherwise prevent or slow the flow of blood into it, may vent through the flexible sleeve 150 and the venting member 160 through the venting member base 300 to the ambient within the guide tube 116. The venting member base 300 may provide a surface for air to escape from the venting member 160. Blood that flows into the blood drawing device may be viewed either through the central body 100, the butterfly connection tube 182 (in embodiments that use a butterfly needle) or through a transparent or translucent flexible sleeve 150 and transparent or translucent guide tube 116. The visual detection of blood indicates positive vein entry, known as flash. Once flash is detected, a fluid sample container may be pressed into the open end 118 of the guide tube 116, the rear cannula 140 may pierce the flexible sleeve 150 and the flexible sleeve is pushed back towards the base of the second rear cannula.

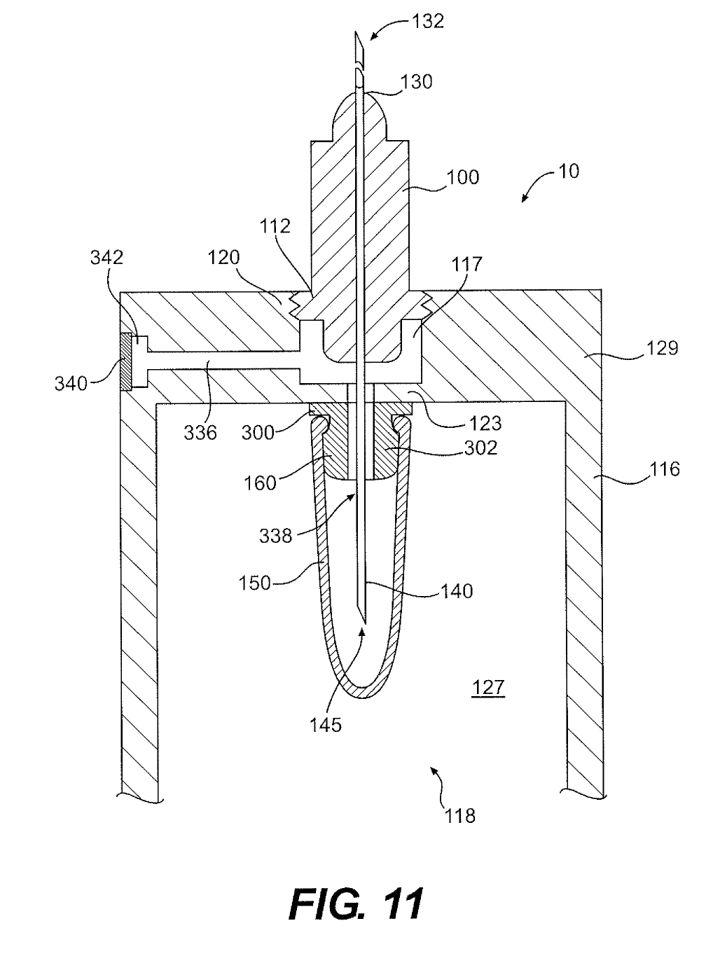

With reference to FIG. 11, another embodiment of the present invention is shown in which all reference characters refer to like elements shown in the previous drawing figures. The embodiment shown in FIG. 11 is identical in all respects to that shown in FIG. 10 with the following exception. With respect to FIG. 11, a well passage 336 extends from the well 117 to the aperture 342. After air vents through the venting member 160, blood may flow from the rear cannula 140 up through the central well opening 338 and into the well 117. The blood may then flow through the well passage 336 and to the viewing window 340. Blood may be viewed either through the central body 100, the butterfly connection tube 182 (in embodiments that use a butterfly needle), through a transparent or translucent flexible sleeve 150 and transparent or translucent guide tube 116, or through the optional viewing window 340. The well 117 may be formed with sufficient space to permit blood to flow to the viewing window without creating significant air back-pressure in the well passage 336 and aperture 342.

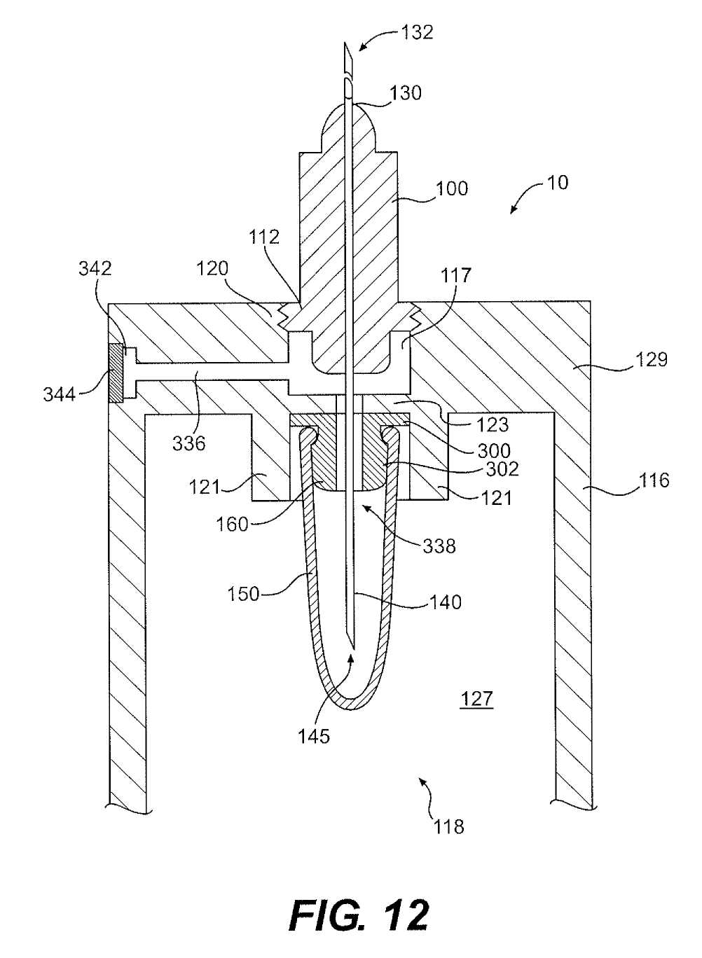

With reference to FIG. 12, another embodiment of the present invention is shown in which all reference characters refer to like elements shown in the previous drawing figures. The embodiment shown in FIG. 12 is identical in all respects to that shown in FIG. 11 with the following exception. With respect to FIG. 12, one or more well bottom extensions 121 may extend from the well bottom wall 123. The well bottom extensions 121 may surround the venting member 160 in whole or in part and may be formed so as to leave an air space for air to flow from the interior of the sleeve 150 through the venting member 160 to the ambient in the guide tube opening 118. The well bottom extensions 121 may be semi-circular or prong shaped and surround the venting member 160. Further, the well bottom extensions 121 may extend into the guide tube 116 a distance sufficient to protect the venting member 160 from being impacted against, damaged or dislodged by, the stopper of a container inserted into the guide tube. The well bottom extensions 121 may be formed of plastic material which is integral with the rest of the guide tube 116 and well bottom wall 123. The well bottom extensions 121 may also be used to secure the venting member 160 and more particularly, the base portion 300 of the venting member so that the venting member is retained in place without the need to use an adhesive or other connection means to the guide tube 116.

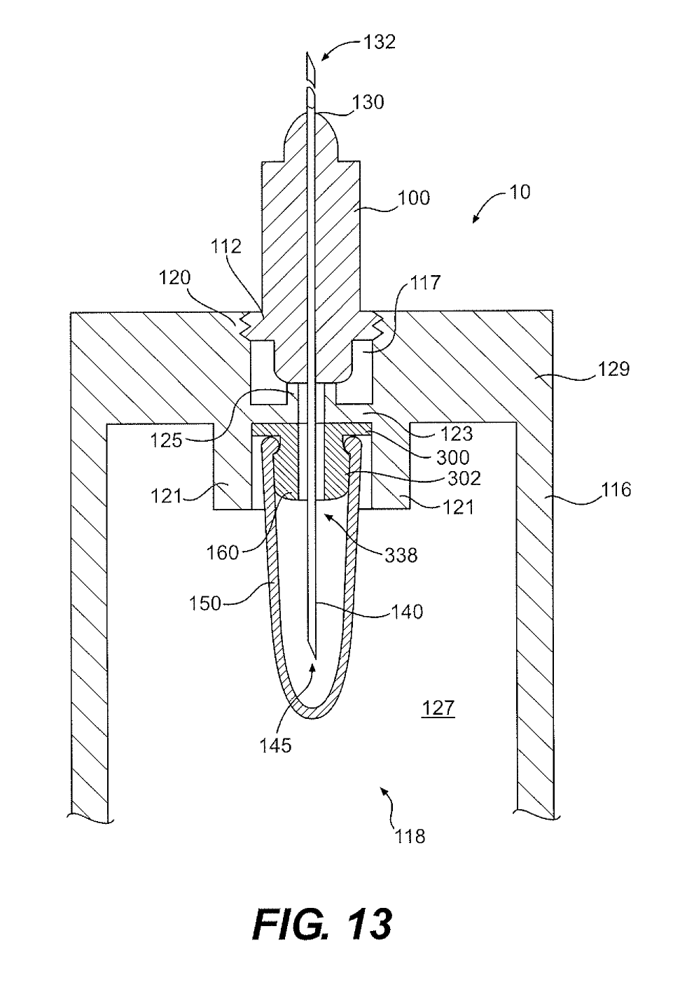

With reference to FIG. 13, another embodiment of the present invention is shown in which all reference characters refer to like elements shown in the previous drawing figures. The embodiment shown in FIG. 13 is identical in all respects to that shown in FIG. 10 with the following two exceptions. With respect to FIG. 13, one or more well bottom extensions 121 may extend from the well bottom wall 123. The well bottom extensions 121 may surround the venting member 160 in whole or in part and may be formed so as to leave an air space for air to flow from the interior of the sleeve 150 through the venting member 160 to the ambient in the guide tube opening 118. The well bottom extensions 121 may be semi-circular or prong shaped and surround the venting member 160. Further, the well bottom extensions 121 may extend into the guide tube 116 a distance sufficient to protect the venting member 160 from being impacted against, damaged or dislodged by, the stopper of a container inserted into the guide tube. The well bottom extensions 121 may be formed of plastic material which is integral with the rest of the guide tube 116 and well bottom wall 123. The well bottom extensions 121 may also be used to secure the venting member 160 and more particularly, the base portion 300 of the venting member so that the venting member is retained in place without the need to use an adhesive or other connection means to the guide tube 116. Further, in FIG. 13 the central body 100 may be connected to the guide tube 116 such that the rear portion of the central body seals against the well bottom wall upper surface 125. Such sealing is not required in this or any other embodiments discussed, but is preferred to limit the amount of blood that may flow into the well space 117 if the central body 100 is disconnected from the guide tube 116.

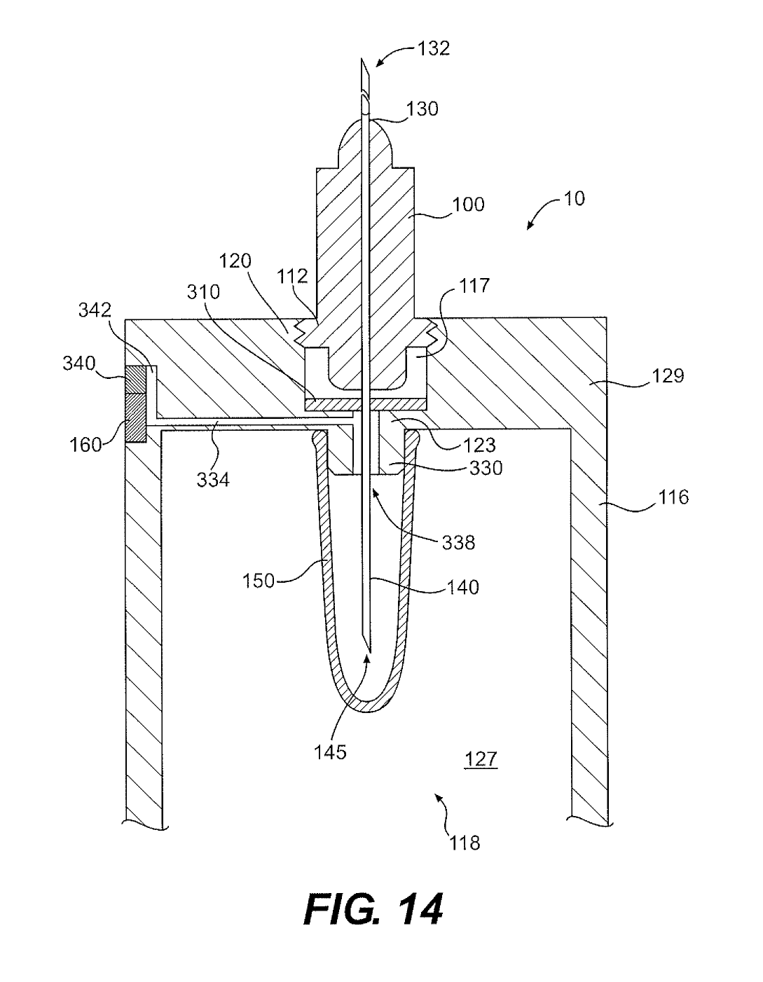

With reference to FIG. 14, another embodiment of the present invention is shown in which all reference characters refer to like elements shown in the previous drawing figures. The well bottom wall 123 may have a sealing membrane 310 made of material similar to or the same as the flexible sleeve 150 which may prevent blood from flowing into the well 117 when the cannula 140 is inserted through and/or withdrawn from it.

With continued reference to FIG. 14, the flexible sleeve 150 may be mounted or fitted onto a sleeve connector 330 which is integrally formed with the guide tube 116 within the guide tube interior opening 127. A central well opening 338 may extend through the sleeve connector 330 and connect with a lateral passage 334 that extends from the central well opening 338 to a guide tube aperture 342 located along the guide tube outer wall. The guide tube aperture 342 may have a venting member 160 disposed in it, and an optional viewing window 340 disposed in it. The viewing window 340 may be transparent or translucent and may permit blood to be viewed by a user through the viewing window so as to detect flash. The viewing window 340 may be disposed next to, circumferentially about, or within the venting member 160 without departing from the intended scope of the invention. In fact, the viewing window 340 and the venting member 160 may be disposed in separate apertures in alternative embodiments. The viewing window 340 also may be unnecessary if flash is detected elsewhere, such as through a translucent or transparent butterfly connection tube, central body, flexible sleeve and/or guide tube. The blood drawing device shown in FIG. 14 may be used in the same manner as that described above in connection with FIG. 8.

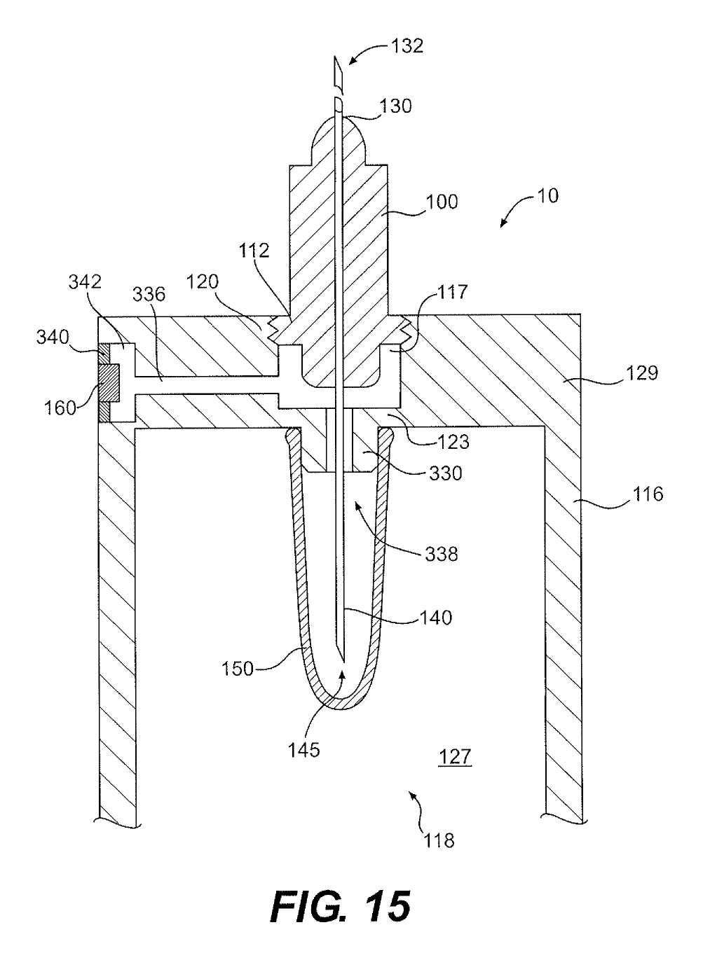

With reference to FIG. 15, another embodiment of the present invention is shown in which all reference characters refer to like elements shown in the previous drawing figures. The embodiment shown in FIG. 15 is identical in all respects to that shown in FIG. 9 with the following exception. With respect to FIG. 15, the central well opening 338 is defined by the well bottom wall 123 and the sleeve connector 330. The second rear cannula (shown in FIG. 9) is not provided. When the front cannula 130 is introduced to the vein of a patient air may vent from the rear cannula 140 up through the central well opening 338 and into the well 117. The air may then flow through the well passage 336 and the venting member 160. After the air is vented, blood may fill the well passage 336 and the aperture 340. Blood may be viewed either through the central body 100, the butterfly connection tube 182 (in embodiments that use a butterfly needle), through a transparent or translucent flexible sleeve 150 and transparent or translucent guide tube 116, or through the optional viewing window 340.

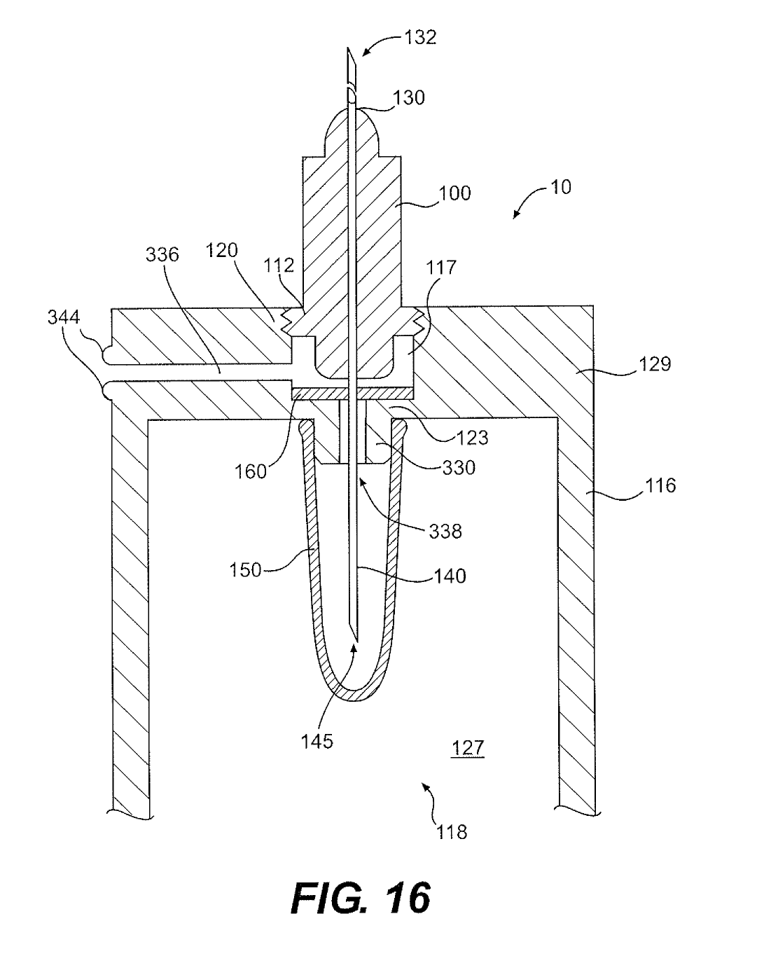

With reference to FIG. 16, another embodiment of the present invention is shown in which all reference characters refer to like elements shown in the previous drawing figures. The flexible sleeve 150 may be mounted or fitted onto a sleeve connector 330 which is integrally formed with the guide tube 116 within the guide tube opening 118. A central well opening 338 may extend through the sleeve connector 330 and connect with a well 117. A venting member 160 which may be a flexible membrane or other porous material may be disposed along the interior of the well bottom wall 123 so as to provide a blood barrier between the central well opening 338 and the well 117. A well passage 336 may extend from the well 117 to the outside of the guide tube 116 outer wall. One or more raised features 344 may be provided adjacent to the end of the well passage 336 to assist a user in tactically sensing the location of the well passage so as not to block it with the user's fingers or hand.

The blood drawing device shown in FIG. 16 may be used as follows. The central body 100 may be connected to the guide tube 116 such that the rear cannula 140 extends through the venting member 160. Once the central body 100 is connected to the guide tube 116, the front cannula 130 or butterfly cannula 184 (in alternative embodiments that utilize a butterfly needle) may be introduced into a lumen, such as a vein, to draw fluid, such as blood. The blood may readily flow through the front cannula 130, the rear cannula 140 and into the interior of the flexible sleeve 150. Air within the blood drawing device 10 that would otherwise prevent or slow the flow of blood into it, may vent through the flexible sleeve 150, the central well opening 338, the venting member 160 and the well passage 336 to the ambient outside the guide tube 116. Blood that flows into the blood drawing device may be viewed either through the central body 100, the butterfly connection tube 182 (in embodiments that use a butterfly needle), or through a transparent or translucent flexible sleeve 150 and transparent or translucent guide tube 116. The visual detection of blood indicates positive vein entry, known as flash. Once flash is detected, a fluid sample container may be pressed into the open end 118 of the guide tube 116, the second rear cannula 141 may pierce the flexible sleeve 150 and the flexible sleeve is pushed back towards the base of the second rear cannula.

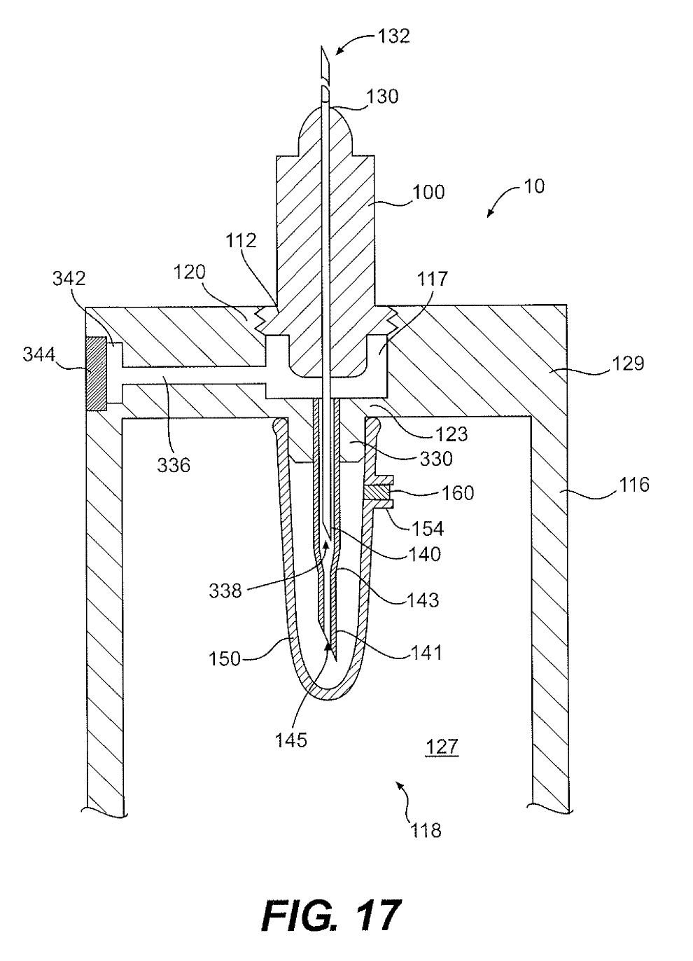

With reference to FIG. 17, another embodiment of the present invention is shown in which all reference characters refer to like elements shown in the previous drawing figures. The flexible sleeve 150 may be mounted or fitted onto a sleeve connector 330 which is integrally formed with the guide tube 116 within the guide tube opening 118. A central well opening 338 may be defined by a second rear cannula 141 and provide fluid communication between the interior of a flexible sleeve 150 and a well 117 provided in the guide tube 116. A well passage 336 may extend from the well 117 to a guide tube aperture 342 located along the guide tube 116 outer wall. The guide tube aperture 342 may have a viewing window 340 disposed in it. The viewing window 340 may be transparent or translucent and may permit blood to be viewed by a user through the viewing window so as to detect flash. The flexible sleeve 150 may include a side tubulation 154 in which a venting member 160 is inserted.

The blood drawing device shown in FIG. 17 may be used as follows. The central body 100 may be connected to the guide tube 116. Once the central body 100 is connected to the guide tube 116, the front cannula 130 or butterfly cannula 184 may be introduced into a lumen, such as a vein, to draw fluid, such as blood. The blood may readily flow through the front cannula 130, the first rear cannula 140, the second rear cannula 141 and into the interior of the flexible sleeve 150. Air within the blood drawing device 10 that would otherwise prevent or slow the flow of blood into it, may vent through the venting member 160 provided in the side tubulation 154 in the flexible sleeve 150 to the ambient inside the guide tube 116. Blood that flows into the blood drawing device may be viewed either through the central body 100, the butterfly connection tube 182 (in embodiments that use a butterfly needle), through a transparent or translucent flexible sleeve 150 and transparent or translucent guide tube 116, or through the optional viewing window 340. The viewing window 340 may be unnecessary if flash is detected elsewhere, such as through a translucent or transparent butterfly connection tube, central body, flexible sleeve and/or guide tube.

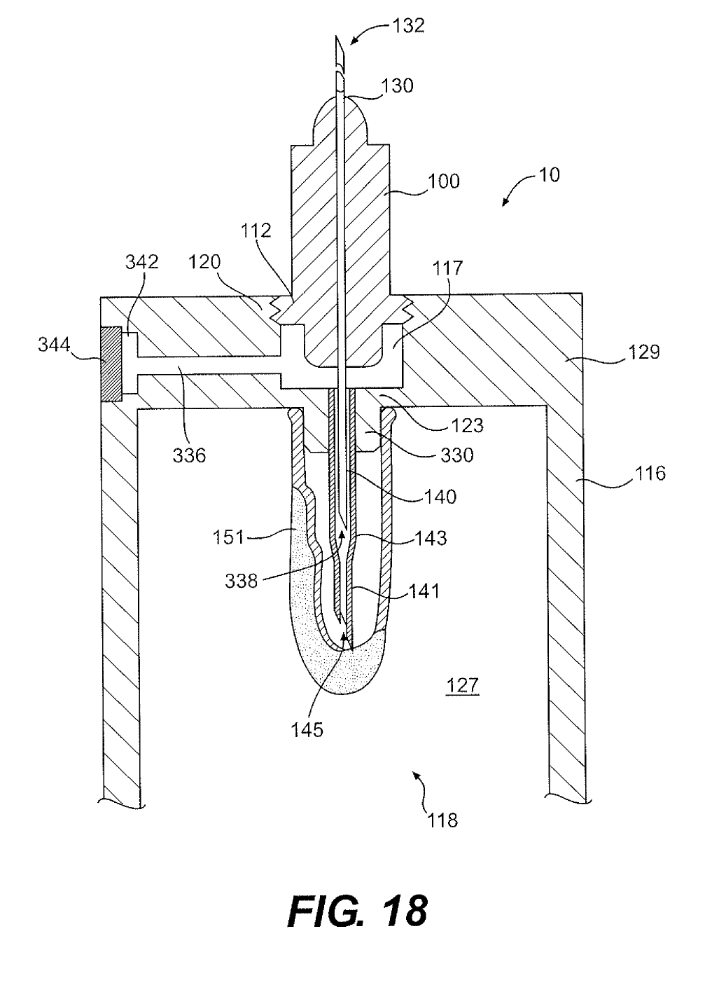

With reference to FIG. 18, another embodiment of the present invention is shown in which all reference characters refer to like elements shown in the previous drawing figures. The embodiment shown in FIG. 18 is identical in all respects to that shown in FIG. 17 with the following exception. In FIG. 18, an air-permeable, blood impermeable flexible sleeve 151 replaces the flexible sleeve 150 shown in FIG. 17. The air-permeable, blood-impermeable sleeve 151 serves as a venting member.

The air-permeable, completely or partially blood-impermeable flexible sleeve 151 may be constructed of a material that is largely air-permeable, but partially, largely or entirely impermeable to blood. The air-permeable sleeve 151 may be used to isolate the rear cannula 140 of a blood drawing device from the ambient in the same manner as conventional sleeve may isolate rear cannulae. During a blood drawing procedure using a device not equipped with a means for venting air from the sleeve, blood from a lumen may be slowed or prevented from entering the device due to air back pressure in the device. In these devices the air in the device may be trapped because there is no vent provided. In the present embodiment, an air-permeable sleeve 151 replaces a conventional sleeve in the guide tube 116. The air-permeable sleeve 151 may provide a pathway to vent air from the device interior, through the sleeve wall, to the ambient. As the air is vented, the blood filling the device may contact the air-permeable sleeve 151. However, the air-permeable sleeve 151 may prevent or retard the flow of blood through its wall because the pore size of the air-permeable sleeve may be large enough to allow the passage of air, but too small to allow much or any blood to pass. This air passage-blood blockage may permit blood to fill the needle and/or the sleeve 151 more readily because there is reduced or no air back pressure inhibiting the flow of blood into the blood drawing device. As a result, a blood drawing device equipped with the air-permeable sleeve 151 may indicate flash (the visual indication of blood flow into the needle) more readily. The air-permeable sleeve 151 may be used with conventional needle drawing or infusion sets (such as butterfly needles), hypodermic needles, or the like, to enhance flash indication.

The air-permeable sleeve 151 may be made of any suitable material that is completely or at least partially air-permeable and substantially blood impermeable, such as for example, low density polyethylene or low density rubber. One example of a method of making such material is described in U.S. Pat. No. 5,641,442. A second example may be made of crumbed material of sufficiently low density/high flexibility to allow the required flexibility in spite of the use of thermal binders like polyethylene. Low density material such as low density silicone may be sifted using a #80 mesh and mixed with #100 mesh low density polyethylene. This mixture may be heated at approximately 280° F. and injected into a cavity mold to form the selectively porous sleeve 151.

An air-permeable sleeve may be constructed of porous material formed from the combination of a hydrophobic porous material with a hydrophilic porous agent. The hydrophobic porous material, for example, may be a polymeric matrix of either thermoplastic resins such as polyvinyl chloride or copolymers thereof, or synthetic or natural thermosetting rubber-like polymers. In a second example, the polymeric matrix may be rubber-like polymers combined with additives such as anti-degradants, cross-linking agents, cure inhibitors, platinum and other type catalysts, inert fillers, or like materials used to compound thermosetting compounds, and intimately mixed with a hydrophilic porous agent such as silica hydrogel, precipitated hydrated silica, for example such as that sold under the trademark Hi-Sil from PPG Industries, or polyacrylamide gel, cross-linked homopolymer of acrylamide, for example such as that sold under the trademark Agrosoake from Agrosoake International, inert fillers and/or water or solvent soluble porosics. In a third example, the polymeric matrix may be made of a synthetic or natural thermosetting polymer or copolymer, such as those that may be made in accordance with the methods disclosed in U.S. Pat. No. 4,548,835 to Takahashi, et al. and U.S. Pat. No. 4,153,760 to Sundberg et al, for example, each of which is hereby incorporated by reference.

The porous agent may be prepared by polymerizing acrylamide in the presence of an aqueous sodium carbonate to produce a partially hydrolyzed, lightly cross-linked, polyacrylamide gel in accordance with the method disclosed in U.S. Pat. No. 3,022,279 to Proffitt, for example, which is hereby incorporated by reference. The polyacrylamide gel may be produced in bead or granular form using an inverse suspension polymerization method for water-soluble monomer, which is disclosed in U.S. Pat. No. 2,982,749 to Friedrich et al., for example, and which is hereby incorporated by reference.

In one embodiment, for example, the hydrophilic granules may be added to the hydrophobic material in sufficient quantities to create a hydrophilic/hydrophobic porous material. The porosity of the hydrophobic material may be manifested by a network of voids/pores extending throughout the matrix or binder, between neighboring particles of the dispersed filler and portions of the polymeric matrix, which may be achieved by the shrinking of the swollen hydrophilic granules during the dehydration/curing phase. The resultant degree of porosity may be controlled by the amount of water or water substitute added to the polymeric matrix binder material during the mixing phase, the vulcanization of the polymeric matrix (such as for example, under hydrostatic conditions in a steam autoclave to a state of cure using the pressurized steam as a source of heat), the proportion and size of the hydrophilic granules added, the duration of the mixing phase, and the wall thickness of the elastomeric sleeve. The hydrophilic granules may be mixed with a normally hydrophobic binder (and water or a water substitute may be added to control porosity) in a mixing type extruder.

When this material is formed into an air-permeable flexible sleeve 151, water-based liquids such as blood may rapidly soak into the pores/voids containing the granular material, causing the granules to swell and seal the pores/voids contained within the polymeric matrix. Thus, the air-permeable flexible sleeve, which is initially permeable to air, may become relatively impermeable to liquids, such as blood, due to the swelling of the moisture reactive granules entrapped within the pores/voids within the polymeric matrix.

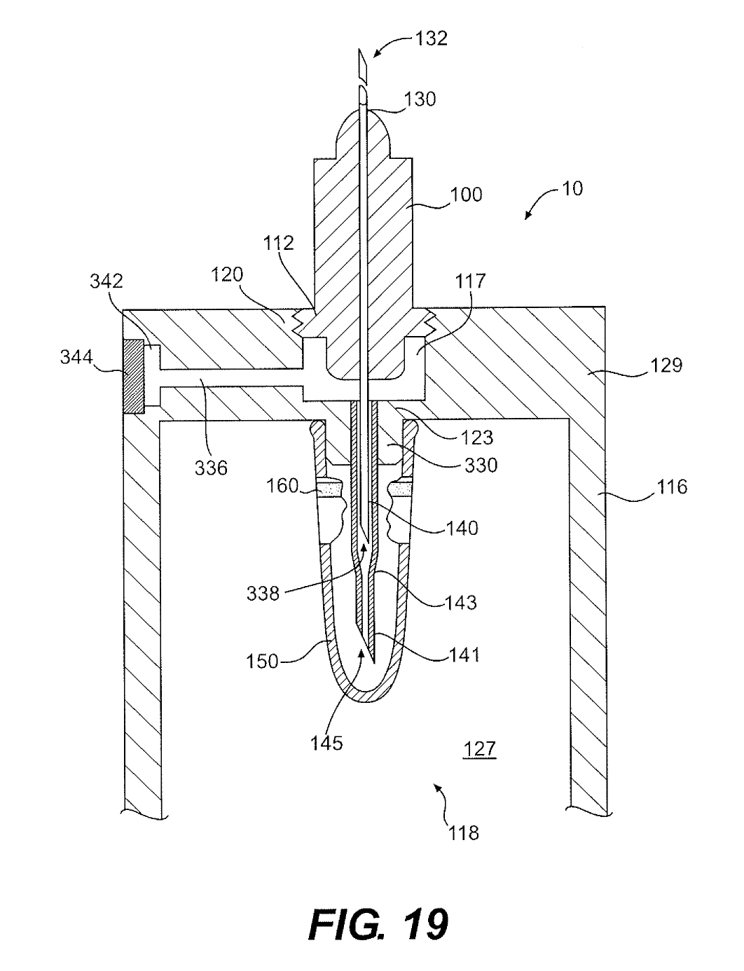

With reference to FIG. 19, another embodiment of the present invention is shown in which all reference characters refer to like elements shown in the previous drawing figures. The embodiment shown in FIG. 19 is identical in all respects to that shown in FIG. 17 with the following exception.

In the embodiment shown in FIG. 19, a portion of the flexible sleeve 150 may be made to be porous to air. A challenge associated with making the flexible porous sleeve 150 shown in FIG. 19 is that many methods of making a material porous may render the material less flexible. For example, porous “soaker” hoses are made by mixing ground rubber with a thermoplastic such as polyethylene which allows the ground rubber to be “sintered” or stuck together by melting the polyethylene. The addition of the polyethylene makes the hose much stiffer than a plain rubber hose. The same may be true when making a fully porous multiple sample sleeve as shown in FIG. 18—the resulting sleeve may be less flexible than required. As a result, one solution is to restrict the porous portion of the flexible sleeve 150 to the area near but not all the way at the open end of the sleeve as shown in FIG. 19, so that air can vent from the sleeve while still permitting the open end of the sleeve and the closed end of the sleeve to be made of relatively more flexible material which does not vent. This way the open end of the sleeve may be flexible enough to be stretched over the sleeve connector 330 and the closed end of the sleeve may be flexible enough to be pierced by the second rear cannula 141 without breaking apart while reverting to its original shape after being compressed by the vacuum tube after its removal. This ability to maintain the sleeve's compressibility and “memory” function may be important to the proper functioning of the sleeve.

With reference to FIG. 19, the flexible sleeve 150 may be made of a first type of flexible sleeve material which does not substantially vent air (i.e., is the air-impermeable portion of the sleeve) such as the material described in U.S. Pat. No. 3,877,465 to Miyake, for example, and of a second type of flexible sleeve material that makes up the venting member 160 which is air-permeable and completely or partially blood-impermeable (i.e., the air-permeable blood-impermeable portion of the sleeve) and formed as a venting port or wall portion added to or incorporated or integrated into the sleeve 150. The venting member 160 may be of any shape or size suitable for permitting air to vent from the interior of the sleeve 150 while completely or substantially preventing the passage of blood. The venting member 160 may be made of some flexible material other than just air, meaning that the venting member 160 is not merely an opening, but rather a second type of material having numerous small air passages as distinguished from the first type of material that makes up the flexible sleeve 150 or a single larger opening or hole in the first type of material.

In the embodiment of the invention shown in FIG. 19, the venting member 160 may be constructed of a material that is largely air-permeable, but partially, largely, or entirely impermeable to blood, and preferably made of similar or the same material as used to construct the sleeve 151 shown in FIG. 18. Preferably, the venting member 160 may be provided as near to the open end of the flexible sleeve 150 as is practically possible, however, in alternative embodiments, the venting member may be placed in alternate locations closer to or near the closed end of the flexible sleeve 150.

The porous venting member 160 shown in FIG. 19 could be achieved by forming a part of the sleeve wall by partially hydrating an absorbent such as polyacrylamide gel with water and mixing it with a heat cured injection molding silicone. When subjected to the typical curing temperatures of 120-200° degrees Celsius the water may convert to steam once the sleeve is ejected from the mold. This may create an open cell structure which contains granules of polyacrylamide. These granules may be processed further during a post cure phase where the granules are further dehydrated with heated dry air. This drying process may create open spaces around each granule which allows air to escape. These same granules absorb water and swell when exposed to blood, thereby sealing the venting portion to prevent leakage. With particular reference to FIG. 19, the resulting porous blood-impermeable air-permeable ring shaped venting member 160 can be inserted into sleeve molds where transparent silicone can be injected adjacent to both edges of the venting port, thus bonding the venting portion with the transparent, more flexible body portion material of the sleeve.

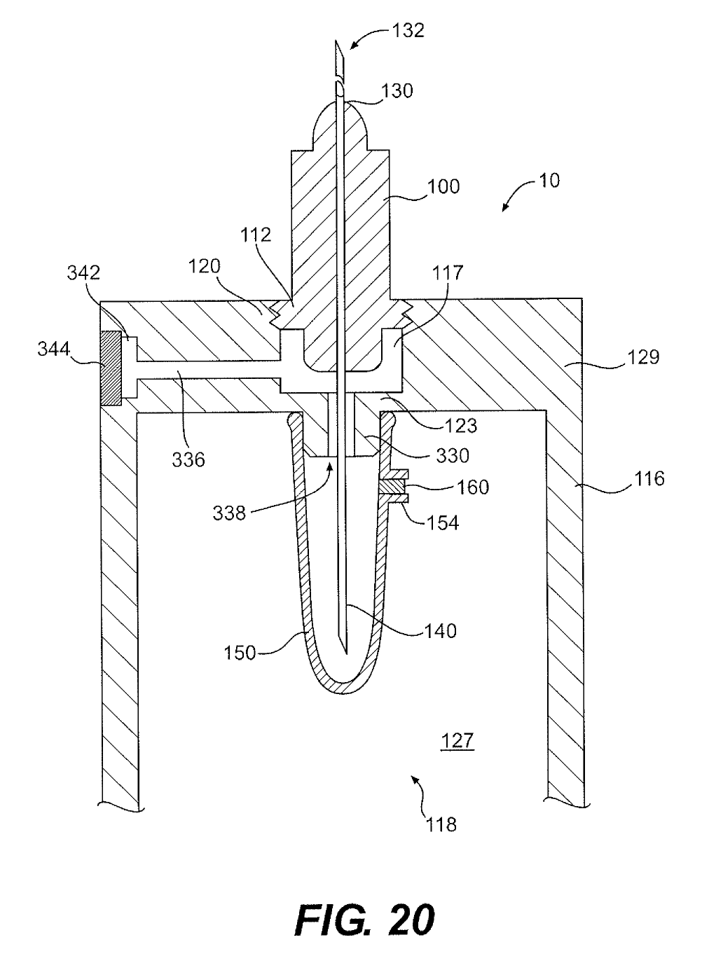

With reference to FIGS. 20-22, three other embodiments of the present invention are shown in which all reference characters refer to like elements shown in the previous drawing figures. The embodiments shown in FIGS. 20-22 are identical in all respects to those shown in FIGS. 17-19, respectively, with the following exception. With respect to FIGS. 20-22, the central well opening 338 is defined by the well bottom wall 123 and the sleeve connector 330. The second rear cannula (shown in FIGS. 17-19) is not provided. When the front cannula 130 is introduced to the vein of a patient air may vent from the rear cannula 140 through the venting members 160 or air-permeable, blood-impermeable flexible sleeve 151 shown in FIGS. 20-22. After the air is vented, blood may fill the well passage 336 and the aperture 340. Blood may be viewed either through the central body 100, the butterfly connection tube 182 (in embodiments that use a butterfly needle), through a transparent or translucent flexible sleeve 150 and transparent or translucent guide tube 116, or through the optional viewing window 340. As noted above, the size of the well 117 may be sufficient to permit blood to fill the aperture 342 without developing sufficient air back-pressure to prevent such filling.

It is appreciated that the embodiments of the invention shown in FIGS. 1-24 may be altered as follows without departing from the intended scope of the invention. The shoulder 119, the dimples 217 and the radial lines 219 on the bottom of the well 117 could be replaced by a shoulder, raised dimples or raised radial lines on the surface of the venting member 160 closest to the well bottom wall 123. Accordingly, the illustrations provided in FIGS. 1, 4 and 5 of the shoulder 119, the dimples 217 and the radial lines 219 are intended to also illustrate such features as they may be provided on a venting member.

With regard to FIGS. 23-24, the butterfly needle 180 may be connected to the Luer-type hub 102 via a butterfly connection tube 182. The butterfly needle 180 may include a butterfly (i.e., front) cannula 184 and one or more wings 186. The butterfly cannula 184 may be inserted directly into the body lumen for blood collection. Flash may be observed in the transparent or translucent butterfly connection tube 182, in which case the central body 100 need not be transparent or translucent (although it could be).

With continued reference to FIGS. 23-24, known butterfly needles may use a butterfly connection tube 182 approximately 12 or more inches in length. This length of tubing is used so as to provide a sufficiently long column of air to permit flash observation when the blood-drawing device 10 is not provided with an air vent. Specifically, when a butterfly connection tube is used without an air vent, the flow of fluid through the butterfly needle may compress the volume of air in the butterfly connection tube 182, the fluid passage 110, the rear cannula 140, and the space between the rear cannula and the flexible sleeve 150. Because there is no vent provided, as blood flows into the device, the air in the device exerts an increasing level of backpressure on the blood, which may prevent blood flow and flash detection. The inclusion of a butterfly connection tube approximately 12 inches in length or greater increases the relative volume of air in the blood collection device. The increased volume of air in the device may permit flash detection before the air backpressure in the device rises to a level that prevents further blood flow into the device and could frustrate flash detection. Butterfly connection tubes of this length may be coiled in packaging, and retain some coil memory after they are removed from their packaging. Previously coiled butterfly connection tubes may resist being straightened for use and have an inherent bias towards returning to their coiled shape. Accordingly, manipulation of a butterfly needle attached to a previously coiled butterfly connection tube may be difficult due to the connection tube's tendency to recoil. This action can be the cause of accidental needle sticks for the healthcare worker and the patient. Furthermore, the coil memory of the tubing may make handling generally difficult for lumen insertion, and/or maintenance of the needle in the lumen.

The butterfly connection tube 182 used in the devices shown in FIGS. 23-24 may be less than approximately 12 inches in length, and more preferably, may be only a few inches in length as a result of the inclusion of a venting member 160 in the blood-drawing device 10. The inclusion of the venting member 160 may obviate the need for a relatively long column of air in the butterfly connection tube that otherwise may be needed to indicate flash. The use of a shortened butterfly connection tube 182 may also obviate the need to coil the tube prior to use, thereby eliminating the issues associated with coil memory in the tube, as well as make it possible to use rigid or semi-rigid connection tubes that may better enable placement of the front cannula into the body lumen.

With particular reference to FIG. 24, the need for a well (discussed above in connection with FIGS. 1-22) may be eliminated by directly connecting the Luer-adapter 102 to a mating adapter 103 which is integrally formed with the guide tube 116.

Each of the embodiments of the present invention shown in all of the afore-noted figures may also utilize a transparent or translucent flexible sleeve 150 to provide flash detection. An example of a transparent sleeve is disclosed in U.S. Pat. No. 3,886,930 to Ryan, which is hereby incorporated by reference. Use of a transparent or translucent sleeve 150 may make it unnecessary for the central body 100 or other elements of the device to be constructed of transparent or translucent material because the flash may be detected through the wall of the sleeve itself and thereby allow for the retrofitting of known blood-drawing devices to provide air venting and flash detection without other modification of the device. Use of a transparent or translucent sleeve 150 may also obviate the need to have discreet front and rear cannulae 130 and 140. The front and rear cannulae may be constructed from a single integral piece of material because in this embodiment of the invention there may be no need to view flash in the central body 100.

Each of the embodiments of the invention described above may also be modified such that the porous member 160 includes or is constructed of any one or more of a number of substances that may permit air venting, and limit and reduce blood seepage, but not completely prevent blood seepage through the particular porous structure. For example, hydrophilic and/or hydrophobic substances such as polyethylene and granular starch, cellulose, polyacrylamide gel, or the like may be used. Such substances are known in the art, and may be used to permit gas (e.g., air) to flow through them, but absorb or block liquid substances. Accordingly, a porous member comprised of these materials may be used to permit the air in a blood drawing device to vent past it until it is contacted by a liquid, such as blood, at which time the blood may be absorbed.

Similarly, glass powder or fiber may be used to simulate clotting, or a clotting agent, such as dilute Russell Viper Venom, may be used to permit air venting with little or reduced blood seepage. Russell Viper Venom is known in the art as a clotting agent. A porous member 160 impregnated with a clotting agent or simulating clotting agent may be used to permit the air in a blood drawing device to vent until it is contacted by blood, at which time the blood may clot or act as clotted and reduce further blood seepage through the porous member. As a result, use of hydrophilic and/or clotting agents in the previously described porous member may permit improved blood flow into a blood drawing device and flash detection.

It will be apparent to those skilled in the art that variations and modifications of the present invention can be made without departing from the scope or spirit of the invention. For example, the shape, size, and material selection for the various components of the blood-drawing device may be changed without departing from the intended scope of the invention and appended claims. It is further appreciated that forming one or more elements of the apparatus embodiments of the present invention integrally as opposed to separately is intended to fall within the scope of the invention and appended claims.

What is claimed is:

1. A guide tube assembly for connection to a device for drawing fluid from a lumen, comprising:
a guide tube having an open end and a well;
a flexible sleeve having an interior space in fluid communication with the well and extending into the guide tube;
one or more passages extending through the guide tube which are in fluid communication with the flexible sleeve interior space; and
a venting member connected to the guide tube or incorporated into the flexible sleeve, said venting member in fluid communication with the one or more passages and disposed between the flexible sleeve interior space and an ambient.

2. The guide tube assembly of claim 1, wherein:
the guide tube has an interior opening and a substantially closed end;
the well is defined by a well bottom wall and is formed in the substantially closed end of the guide tube;
one of the one or more passages is a central well opening extending from the well to the guide tube interior opening;
the venting member is connected to the guide tube within the guide tube interior opening;
the flexible sleeve is mounted on the venting member and extends into the guide tube interior opening, said sleeve defining an interior space; and
one or more well bottom extensions extend into the guide tube interior opening adjacent to the venting member.

3. The guide tube of claim 2 wherein the venting member has a base exposed to the ambient and a barb portion adapted to receive the flexible sleeve.

4. The guide tube of claim 3 wherein the venting member is adhered to the well bottom wall.

5. The guide tube of claim 3 wherein the venting member contacts raised dimples provided on the well bottom wall.

6. The guide tube of claim 3 wherein the venting member contacts a raised shoulder provided on the well bottom wall.

7. The guide tube of claim 3 wherein the venting member contacts raised radial lines provided on the well bottom wall.

8. The guide tube of claim 2 wherein the central well opening is defined by a rear cannula connected to the guide tube and extending into the guide tube interior opening.

9. The guide tube of claim 8 wherein the rear cannula has a stepped diameter.

10. The guide tube of claim 2 further comprising a well bottom wall upper surface adapted to seal against a central body of a blood drawing device.

11. The guide tube of claim 2 wherein the one or more well bottom extensions are adapted to retain the venting member by a frictional fit.

12. The guide tube of claim 2 wherein the one or more well bottom extensions extend an equal to greater distance into the guide tube interior opening than the venting member.

13. The guide tube of claim 2 further comprising:
a well passage extending from the well to an aperture provided in a side wall of the guide tube; and
a viewing window disposed in the aperture.

14. The guide tube of claim 2 wherein the guide tube and the flexible sleeve are transparent or translucent.

15. The guide tube assembly of claim 1, wherein:
the guide tube has an interior opening and a substantially closed end;
the well is defined by a well bottom wall and is formed in the substantially closed end of the guide tube;
one of the one or more passages is a central well opening extending from the well to the guide tube interior opening;
the venting member is connected to the guide tube within the guide tube interior opening, said venting member having a base and a barb portion; and
the flexible sleeve is mounted on the venting member and extends into the guide tube interior opening, said sleeve defining an interior space and being mounted on said venting member so that the base of the venting member is exposed to an ambient.

16. The guide tube of claim 15 wherein the venting member is adhered to the well bottom wall.

17. The guide tube of claim 15 wherein the central well opening is defined by a rear cannula connected to the guide tube and extending into the guide tube interior opening.

18. The guide tube of claim 17 wherein the rear cannula has a stepped diameter.

19. The guide tube of claim 15 further comprising a well bottom wall upper surface adapted to seal against a central body (100) of a blood drawing device.

20. The guide tube of claim 15 further comprising:
a well passage extending from the well to an aperture provided in a side wall of the guide tube; and
a viewing window disposed in the aperture.

21. The guide tube of claim 15 wherein the guide tube and the flexible sleeve are transparent or translucent.

22. The guide tube assembly of claim 1 further comprising:
a sleeve connector extending from a well bottom wall into a guide tube interior opening, and wherein
the guide tube has a substantially closed end;
the well is defined by the well bottom wall and is formed in the substantially closed end of the guide tube;
one of the one or more passages is a central well opening extending from the well to the guide tube interior opening through the sleeve connector;
the flexible sleeve is mounted on the sleeve connector and extends into the guide tube interior opening, said sleeve defining an interior space; and
the one or more passages connect the sleeve interior space with an aperture provided in a wall of the guide tube.

23. The guide tube of claim 22 wherein the venting member is disposed in the aperture.

24. The guide tube of claim 23 further comprising a viewing window disposed in the aperture.

25. The guide tube of claim 24 wherein the viewing window is disposed within the venting member.

26. The guide tube of claim 24 wherein the viewing window is disposed about the venting member.

27. The guide tube of claim 24 wherein the viewing window is disposed adjacent to the venting member.

28. The guide tube of claim 22 further comprising a viewing window disposed in the aperture.

29. The guide tube of claim 22 further comprising:
a second aperture in the guide tube wall communicating with the one or more passages; and
a viewing window disposed in the second aperture.

30. The guide tube of claim 22 wherein the central well opening is defined by a rear cannula connected to the guide tube and extending into the guide tube interior opening.

31. The guide tube of claim 30 wherein the rear cannula has a stepped diameter.

32. The guide tube of claim 22 further comprising a well bottom wall upper surface adapted to seal against a central body of a blood drawing device.

33. The guide tube of claim 22 further comprising a sealing membrane disposed in the well along the well bottom wall.

34. The guide tube of claim 22 wherein the one or more passages communicate with the well.

35. The guide tube of claim 22 wherein the venting member is disposed in the well along the well bottom wall.

36. The guide tube of claim 22 wherein the aperture is surrounded by one or more raised features on the wall of the guide tube.

37. The guide tube of claim 22 wherein the venting member is disposed in a tubulation provided in the side wall of the flexible sleeve.

38. The guide tube of claim 37 further comprising a viewing window disposed in the aperture.

39. The guide tube of claim 37 wherein the central well opening is defined by a rear cannula connected to the guide tube and extending into the guide tube interior opening.

40. The guide tube of claim 39 wherein the rear cannula has a stepped diameter.

41. The guide tube of claim 22 wherein the venting member comprises an air-permeable sleeve or is integrated into a portion of the flexible sleeve.

42. The guide tube of claim 41 further comprising a viewing window disposed in the aperture.

43. The guide tube of claim 41 wherein the central well opening is defined by a rear cannula connected to the guide tube and extending into the guide tube interior opening.

44. The guide tube of claim 43 wherein the rear cannula has a stepped diameter.

45. The guide tube of claim 22 wherein the guide tube and the flexible sleeve are transparent or translucent.

46. The device of claims 2, 15 and 22 wherein the venting member is permeable to air and impermeable to blood.

47. The device of claim 1, further comprising:
a first connector of a Luer-adapter connected to the guide tube; and
a butterfly needle connected to the first connector of the Luer-adapter by a second connector.

48. The device of claim 1, further comprising:
a first connector of a Luer-adapter integrally formed in an end of the guide tube opposite the guide tube open end; and
a butterfly needle connected to the first connector of the Luer-adapter by a second connector.
